(19) United States
(12) Patent Application Publication
(10) Pub. No.: US 2020/0199255 A1
(43) Pub. Date: Jun. 25, 2020

(54) MULTIVALENT FRAGMENTS OF ANTIBODY 3E10 AND METHODS OF USE THEREOF

(71) Applicants: Yale University, New Haven, CT (US); The United States Government as represented by the Department of Veterans Affairs, Washington, DC (US)

(72) Inventors: James E. Hansen, New Haven, CT (US); Richard H. Weisbart, Los Angeles, CA (US); Philip W. Noble, Windsor (GB)

(21) Appl. No.: 16/676,076

(22) Filed: Nov. 6, 2019

Related U.S. Application Data
(63) Continuation of application No. 15/507,324, filed on Feb. 28, 2017, now abandoned.
(60) Provisional application No. 62/043,228, filed on Aug. 28, 2014.

(51) Int. Cl. C07K 16/44; A61K 45/06; A61K 39/395

(52) U.S. Cl. CPC C07K 16/44; A61K 45/06; A61K 39/39583; C07K 2319/80; C07K 2319/09; A61K 2039/505; C07K 2317/626; C07K 2317/624; C07K 2317/565; C07K 2317/56; C07K 2317/35; C07K 2317/76

(57) ABSTRACT

Antigen binding molecules that bind to the epitope of 3E10, and methods of use thereof are provided. The antigen binding molecule can include, for example, two or more variant single chain variable fragments (scFv) of monoclonal antibody 3E10, wherein the variant scFv has one or more insertions, deletions, or substitutions relative to a corresponding 3E10 scFv, and wherein the molecule can bind, preferably specifically bind, to the epitope of 3E10. Methods of using the antigen binding molecules for treating cancer and viral infections or preventing viral infections are also provided.

### CROSS REFERENCE TO RELATED APPLICATIONS

This application claims benefit of and priority to U.S. Provisional Patent Application No. 62/043,228 filed on Aug. 28, 2014, and where permissible is hereby incorporated by reference in its entirety.

### REFERENCE TO SEQUENCE LISTING

The Sequence Listing submitted as a text file named “YU_6284_ST25.txt,” created on Aug. 26, 2015, and having a size of 23,485 bytes is hereby incorporated by reference.

### FIELD OF THE INVENTION

The field of the invention is generally related to cell-penetrating antibody fragments, particular di-, tri-, and multivalent single chain variable fragments of 3E10, and variants thereof, and methods of use thereof for targeted therapy of DNA-repair deficient malignancies.

### BACKGROUND OF THE INVENTION

Rational design of targeted cancer therapies entails development of safe and specific modulators of tumor targets. The unrivaled specificity of binding by antibodies to their antigens gives them a compelling therapeutic advantage over other molecules that have significant off target effects. However, most antibodies do not cross plasma membranes and cannot directly impact intracellular processes. Therefore, the fact that many tumor-specific targets are sequestered inside cells and nuclei and are inaccessible to most antibodies has proved a limiting factor in antibody-based cancer therapy. Only seven antibodies are presently FDA-approved for the treatment of solid tumors, and all target extracellular antigens (EGFR, HER2, CTLA-4, or VEGF) (Scott, et al., Nature Reviews Cancer, 12:278-287 (2012)).

Select lupus autoantibodies have the unusual capacity to penetrate into cells. A new paradigm in antibody-based cancer therapy in which cell-penetrating lupus autoantibodies are used to disrupt key intracellular processes to selectively effect cancer cells is emerging. The nuclear-localizing lupus anti-DNA autoantibody 3E10 is a prototype that exemplifies this approach. 3E10 penetrates into cell nuclei where it binds DNA and inhibits DNA repair in a manner that is not toxic to normal cells but is synthetically lethal to tumor cells with defective DNA repair due to BRCA2-deficiency (Hansen, et al., Science Translational Medicine, 4:157ra142 (2012)). This finding provided proof of principle for the use of 3E10 against DNA repair-deficient tumors. Antibodies that target membrane antigens on cancer cells require the Fc fragment to activate complement and induce antibody-dependent cell-mediated cytotoxicity (ADCC). By contrast, the Fc region may not contribute any therapeutic advantage to cell-penetrating antibodies such as 3E10 that impact intracellular and intranuclear targets, but instead may be detrimental due to systemic effects of ADCC and complement.

A 3E10 single chain variable fragment with a D31N mutation in CDR1 that penetrates nuclei and binds DNA with greater efficiency than the original antibody was generated, but still had only a modest effect on BRCA2-deficient cancer cells even at high doses (Hansen, et al., Science Translational Medicine, 4:157ra142 (2012), Weisbart, et al., J. Autoimmun., 11:539-546 (1998), Weisbart, et al., Int. J. Oncol., 25:1867-1873 (2004)). The scFv by itself was also not significantly toxic to PTEN-deficient U251 glioma cells (Hansen, et al., Science Translational Medicine, 4:157ra142 (2012), Puc, et al., Cell Cycle, 4:927-929 (2005), Bassi, et al., Science, 341:395-399 (2013)).

Accordingly, there remains a need for improved 3E10-based compositions.

Therefore, it is an object of the invention to provided 3E10-based agents with increased potency, increased efficacy, reduced toxicity, or a combination thereof compared to monoclonal 3E10 and/or monovalent 3E10 scFv.

It is another object of the invention to provide method of use thereof for treating cancer cells and/or virally infected and/or virally transformed cells, preferably to kill the cells.

It is another object of the invention to provide methods of increasing chemosensitivity, and/or increasing radiosensitivity of cells with deficiencies in DNA damage repair, particularly cells that are deficient in BRCA2 and/or PTEN pathways.

### SUMMARY OF THE INVENTION

It has now been discovered that di-scFv, tri-scFv, and other di-, tri-, and multivalent antigen binding fragments and fusion proteins of the monoclonal antibody 3E10, and variants thereof, particularly the variant D31N, without being conjugated to any therapeutic protein, are selectively lethal to cancer cells deficient in DNA repair even in the absence of radiation or chemotherapy. Moreover, di-scFv, tri-scFv, and other di-, tri-, and multivalent antigen binding fragments and fusion proteins of the monoclonal antibody 3E10, and variants thereof, particularly the variant D31N, enhance cancer cell radiosensitivity and chemosensitivity. This effect is potentiated in cells deficient in DNA repair. The Examples below show that a 3E10 di-single chain variable fragment with D31N mutations in CDR1 of the V_{H} region (di-scFv 3E10 (D31N)) has a significantly enhanced impact on BRCA2-deficient cancer cells when compared to the corresponding monovalent scFv, and that di-scFv 3E10 (D31N) suppresses the growth of BRCA2-deficient tumors in vivo. The Examples also show that di-scFv 3E10 (D31N) is also synthetically lethal to PTEN-deficient cancer cells, and that a 3E10 tri-single chain variable fragment with D31N mutations has an even greater impact on DNA repair-deficient cells than di-scFv 3E10 (D31N).

Therefore, antigen binding molecules that bind to the epitope of 3E10, and methods of use thereof are provided. In the most preferred embodiments, the antigen binding molecule includes (1) two or more variant single chain variable fragments (scFv) of monoclonal antibody 3E10, wherein the variant scFv has one or more insertions, deletions, or substitutions relative to a corresponding 3E10 scFv, and wherein the molecule can bind, preferably specifically bind, to the epitope of 3E10; (2) two or more variant scFv of monoclonal antibody 3E10 including at least 90% sequence identity to the scFv of monoclonal antibody 3E10, wherein the molecule can bind, preferably specifically bind, to the epitope of monoclonal antibody 3E10; or (3) two or more scFv of a humanized variant of monoclonal antibody 3E10 including one, two, three, four, five, or six complementary determining regions (CDRs) of monoclonal antibody 3E10, wherein the molecule can bind, preferably specifically bind, to the epitope of monoclonal antibody 3E10.

In preferred embodiments, the scFv includes a heavy chain variable domain and a light chain variable domain. In some embodiments, the heavy chain variable domain includes one, two, or three CDRs of SEQ ID NO:1 or SEQ ID NO:2. The heavy chain variable domain can include the amino acid sequence of SEQ ID NO:1 or 2. In some embodiments, the light chain variable domain includes one, two, or three CDRs of SEQ ID NO:3. The light chain variable domain can include the amino acid sequence of SEQ ID NO:3.

The two or more scFv can be linked by a linker or linkers, for example a peptide, preferably a peptide including glycines and serines and/or threonines. Therefore, in some embodiments, the molecule includes one or more fusion proteins. For example, the amino acid sequence of the fusion protein can include the amino acid sequence of at least one of the scFv, and the molecule includes two or more of the fusion proteins (e.g., dimers, trimer, etc). For example, the molecule can be a diabody, a tribody, a tetrabody, etc.

In some embodiments, the amino acid sequence of the fusion protein includes the amino acid sequence of at least two, three, or more of the single chain variable fragments. For example, the molecule can be a tandem di-scFv, a tandem tri-scFv, etc. An exemplary di-scFv can have the amino acid sequence of SEQ ID NO:26. An exemplary tri-scFv can have the amino acid sequence of SEQ ID NO:27.

The antigen binding molecule can optionally include or be conjugated to a protein transduction domain, a targeting signal, a nuclear localization signal, or a combination thereof.

Pharmaceutical compositions including the antigen binding molecules and a pharmaceutically acceptable carrier, and methods of use thereof are also provided. Exemplary methods include inhibiting DNA repair in a neoplastic or virally exposed or infected cell by contacting the cell with a pharmaceutical composition including antigen binding molecules. In preferred embodiments, the cell is deficient in DNA damage repair, deficient in maintenance of chromosomal integrity, and/or deficient in protection from genotoxic stress. The cell can have one or more mutations in or abnormal expression of DNA repair genes selected from the group consisting of XRCC1, ADPRT (PARP-1), ADPRTL2, (PARP-2), POLYMERASE BETA, CTPS, MLH1, MSH2, PMS2, p53, p21, PTEN, RPA, RPA1, RPA2, RPA3, XPD, ERCC1, XPF, MMS19, RAD51, RAD51b, RAD51C, RAD51D, DMC1, XRCCR, XRCC3, BRCA1, BRCA2, PALB2, RAD52, RAD54, RAD50, MRE11, NB51, WRN, BLM, KU70, KU80, ATM, ATR, CHK1, CHK2, the FANC family of proteins, FANCA, FANCB, FANCC, FANCD1, FANCD2, FANCE, FANCF, FANCG, FANCL, FANCM, FANCI, FANCJ, FANCN, FANCP, RAD1, and RAD9.

In some embodiments, the cancer cell is defective in DNA damage repair. In particular embodiments, the cancer cell is defective in DNA damage repair due to hypoxia.

In some embodiments, the cancer cell is resistant to radiation or chemotherapy. In particular embodiments, the cancer cell is resistant to radiation or chemotherapy due to hypoxia.

In particularly preferred embodiments, the cell is PTEN deficient, BRCA1 deficient, BRCA2 deficient, or a combination thereof. In some embodiments, the cell is radiation resistant and/or resistant to chemotherapy.

The neoplastic cell can be a cancer cell selected from the group consisting of sarcomas, lymphomas, leukemias, carcinomas and adenocarcinomas, blastomas, germ cell tumors, gliomas, neuroendocrine tumors, melanomas, rhabdoid tumors, embryonal tumors, neuroectodermal tumors, carcinoid tumors, craniopharyngiomas, histiocytomas, medulloepitheliomas, mesotheliomas, multiple myelomas, chronic myeloproliferative disease, primitive neuroectodermal tumors, salivary gland tumors, thymomas, thymic carcinoma, thyroid cancer, and Wilms tumor.

The cell can be part of a hypoxic tumor. The cell can be exposed to or infected with a lentivirus.

The method can include contacting the cell with a radiosensitizer, for example, cisplatin, doxorubicin, gemcitabine, 5-fluorouracil, PARP1 inhibitors, histone deacetylase inhibitors, proteasome inhibitors, epidermal growth factor receptor (EGRF) inhibitors, insulin-like growth factor-1 (IGF-1) receptor inhibitors, CHK1 inhibitors, mTOR inhibitors, kinase inhibitors, pentoxifylline, or vinorelbine. The method can include treating the subject with radiation therapy, wherein the antigen binding molecule increases the cells' sensitivity to radiation therapy.

The method can include treating the subject with a chemotherapeutic or antineoplastic agent. In some embodiments, the antigen binding molecule increases the cells' sensitivity to the antineoplastic drug.

In preferred embodiments, the contacting occurs in vivo in a subject. The subject can have cancer or a viral exposure or viral infection. The pharmaceutical composition can be administered to the subject in an effective amount to reduce or alleviate one or more symptoms of the cancer or the infection. In a particular embodiment, the cancer includes a tumor and the symptom of cancer that is treated is a reduction in tumor burden or a reduction in tumor growth.

Therefore, methods of treating subjects with cancer or a viral infection including administered to the subject an effective amount of a pharmaceutical composition including an antigen binding molecule to reduce or alleviate one or more symptoms of the cancer or infection, or to prevent viral infection after exposure to virus, are also provided.

### BRIEF DESCRIPTION OF THE DRAWINGS

FIGS. 1A and 1B are diagrams showing the domain structure of a scFv 3E10 (D31N) single chain fragment (1A) and di-single chain fragment (1B) of 3E10 (D31N). V_{L}: Variable region of the 3E10 light chain. V_{H}*: Variable region of the 3E10 heavy chain with the enhancing D31N mutation in CDR1.

FIG. 2 is a bar graph showing the surviving fraction of BRCA2+ and BRCA2− cells treated with control buffer or 10 μM scFv 3E10 (D31N) or di-scFv 3E10 (D31N). *p<0.01.

FIG. 3 is a concentration effect curve showing the surviving fraction of BRCA2-deficient CAPAN-1 pancreatic cancer cells treated with 0-6.6 μM scFv 3E10 (D31N) or di-scFv 3E10 (D31N) and evaluated in a colony formation assay.

FIG. 4A is a line graph showing the mean tumor volume over time (days after initial treatment) in a CAPAN-1 subcutaneous xenograft model in nude mice following di-scFv 3E10 (D31N) or buffer control treatment.

FIG. 4B is bar graph showing mean tumor mass (g) of a CAPAN-1 subcutaneous xenograft model in nude mice on day 28 after initial treatment with di-scFv 3E10 (D31N) or buffer control. *p=0.03.

FIG. 5 is a line graph showing the mean body weight (g) of nude mice in a CAPAN-1 subcutaneous xenograft model over time (days) following di-scFv 3E10 (D31N) or buffer control treatment.

FIG. 6 is a diagram showing the domain structure of a tri-scFv 3E10 (D31N) single chain fragment of 3E10 (D31N).

FIG. 7 is a bar graph showing the surviving fraction of BRCA2-deficient CAPAN-1 pancreatic cancer cells treated with control or a low concentration (1 μM) of di-scFv 3E10 (D31N) or tri-scFv 3E10 (D31N) and evaluated in a colony formation assay. *p<0.01.

FIG. 8A is a bar graph showing the mean γH2AX foci/cell of U251 cells after four hours and 24 hours in control media or treatment with 25 μM of scFv 3E10 (D31N) or di-scFv 3E10 (D31N). *p<0.0001. FIG. 8B is a bar graph showing the mean γH2AX foci/cell of U251-PTEN cells after 24 hours in control media or treatment with 25 μM of scFv 3E10 (D31N) or di-scFv 3E10 (D31N). FIG. 8C is a bar graph showing the mean phosphor-53BP1 foci/cell of U251 and U251-PTEN cells after 24 hours in control media or treatment with 20 μM of di-scFv 3E10 (D31N). *p<0.0001.

FIG. 8D is a bar graph showing the surviving fraction of U251 and U251-PTEN cells as determined by colony formation assay after treatment with control media or 3.3 μM of scFv 3E10 (D31N) or di-scFv 3E10 (D31N). *p<0.0001.

FIG. 9A is a bar graph showing the mean γH2AX foci/cell of BRCA2-deficient CAPAN-1 cells after 24 hours in control media or treatment with 25 μM of di-scFv 3E10 (D31N). *p=0.04. FIG. 9B is a concentration-response curve showing the surviving fraction of BRCA2-deficient CAPAN-1 cells following treatment with 0-6.6 μM scFv 3E10 (D31N) or di-scFv 3E10 (D31N) and evaluated by colony formation assay. FIG. 9C is a bar graph showing the surviving fractions of each of an isogenic pair of BRCA2+ and BRCA2− DLD1 cells treated with control buffer or 10 μM scFv 3E10 (D31N) or di-scFv 3E10 (D31N) and evaluated by colony formation assay. *p<0.01. FIG. 9D is a bar graph showing the surviving fractions of each of H1975 (BRCA2-proficient), SW48 (BRCA2-proficient, MLH1-deficient), and HCT116 (BRCA2-proficient, MLH1-deficient) cells treated with control buffer or 3.3 μM scFv 3E10 (D31N) or di-scFv 3E10 (D31N) and evaluated by colony formation assay.

### DETAILED DESCRIPTION OF THE INVENTION

### I. Definitions

As used herein, the term “3E10” refers to a monoclonal antibody produced by ATCC Accession No. PTA 2439 hybridoma.

As used herein, the term “scFv” as used herein means a single chain variable fragment that includes a light chain variable region (V_{L}) and a heavy chain variable region (V_{H}) joined by a linker. The V_{L} and V_{H} regions may be derived from the parent antibody or may be chemically or recombinantly synthesized.

As used herein, the term “variable region” is intended to distinguish such domain of the immunoglobulin from domains that are broadly shared by antibodies (such as an antibody Fc domain). The variable region comprises a “hypervariable region” whose residues are responsible for antigen binding. The hypervariable region comprises amino acid residues from a “Complementarity Determining Region” or “CDR” (i.e., typically at approximately residues 24-34 (L1), 50-56 (L2) and 89-97 (L3) in the light chain variable domain and at approximately residues 27-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e., residues 26-32 (L1), 50-52 (L2) and 91-96 (L3) in the light chain variable domain and 26-32 (H1), 53-55 (H2) and 96-101 (H3) in the heavy chain variable domain; Chothia and Lesk, 1987, J. Mol. Biol. 196:901-917).

As used herein, the term “Framework Region” or “FR” residues are those variable domain residues other than the hypervariable region residues as herein defined.

As used herein, the term “antibody” refers to natural or synthetic antibodies that bind a target antigen. The term includes polyclonal and monoclonal antibodies. In addition to intact immunoglobulin molecules, also included in the term “antibodies” are fragments or polymers of those immunoglobulin molecules, and human or humanized versions of immunoglobulin molecules that bind the target antigen.

As used herein, the term “cell-penetrating anti-DNA antibody” refers to an antibody, or antigen binding fragment or molecule thereof that is transported into the nucleus of living mammalian cells and binds DNA (e.g., single-stranded and/or double-stranded DNA). In preferred embodiments, the antibody is transported into the nucleus of the cells without the aid of a carrier or conjugate. In other embodiments, the antibody is conjugated to a cell-penetrating moiety, such as a cell penetrating peptide.

As used herein, the term “specifically binds” refers to the binding of an antibody to its cognate antigen (for example DNA) while not significantly binding to other antigens. Preferably, an antibody “specifically binds” to an antigen with an affinity constant (Ka) greater than about 10⁵ mol⁻¹ (e.g., 10⁶ mol⁻¹, 10⁷ mol⁻¹, 10⁸ mol⁻¹, 10⁹ mol⁻¹, 10¹⁰ mol⁻¹, 10¹¹ mol⁻¹, and 10¹² mol⁻¹ or more) with that second molecule.

As used herein, the term “monoclonal antibody” or “MAb” refers to an antibody obtained from a substantially homogeneous population of antibodies, i.e., the individual antibodies within the population are identical except for possible naturally occurring mutations that may be present in a small subset of the antibody molecules.

As used herein, the term “DNA repair” refers to a collection of processes by which a cell identifies and corrects damage to DNA molecules. Single-strand defects are repaired by base excision repair (BER), nucleotide excision repair (NER), or mismatch repair (MMR). Double-strand breaks are repaired by non-homologous end joining (NHEJ), microhomology-mediated end joining (MMEJ), or homologous recombination. After DNA damage, cell cycle checkpoints are activated, which pause the cell cycle to give the cell time to repair the damage before continuing to divide. Checkpoint mediator proteins include BRCA1, MDC1, 53BP1, p53, ATM, ATR, CHK1, CHK2, and p21.

As used herein, the term “impaired DNA repair” refers to a state in which a mutated cell or a cell with altered gene expression is incapable of DNA repair or has reduced activity or efficiency of one or more DNA repair pathways or takes longer to repair damage to its DNA as compared to a wild type cell.

As used herein, the term “chemosensitivity” refers to the relative susceptibility of cancer cells to the effects of anticancer drugs. The more chemosensitive a cancer cell is, the less anticancer drug is required to kill that cell.

As used herein, the term “radiosensitivity” refers to the relative susceptibility of cells to the harmful effect of ionizing radiation. The more radiosensitive a cell is, the less radiation that is required to kill that cell. In general, it has been found that cell radiosensitivity is directly proportional to the rate of cell division and inversely proportional to the cell's capacity for DNA repair.

As used herein, the term “radioresistant” refers to a cell that does not die when exposed to clinically suitable dosages of radiation.

As used herein, the term “neoplastic cell” refers to a cell undergoing abnormal cell proliferation (“neoplasia”). The growth of neoplastic cells exceeds and is not coordinated with that of the normal tissues around it. The growth typically persists in the same excessive manner even after cessation of the stimuli, and typically causes formation of a tumor.

As used herein, the term “tumor” or “neoplasm” refers to an abnormal mass of tissue containing neoplastic cells. Neoplasms and tumors may be benign, premalignant, or malignant.

As used herein, the term “cancer” or “malignant neoplasm” refers to a cell that displays uncontrolled growth and division, invasion of adjacent tissues, and often metastasizes to other locations of the body.

As used herein, the term “antineoplastic” refers to a composition, such as a drug or biologic, that can inhibit or prevent cancer growth, invasion, and/or metastasis.

As used herein, the term “anti-cancer moiety” refers to any agent, such as a peptide, protein, nucleic acid, or small molecule, which can be combined with the disclosed anti-DNA antibodies to enhance the anti-cancer properties of the antibodies. The term includes antineoplastic drugs, antibodies that bind and inhibit other therapeutic targets in cancer cells, and substances having an affinity for cancer cells for directed targeting of cancer cells.

As used herein, the term “virally transformed cell” refers to a cell that has been infected with a virus or that has incorporated viral DNA or RNA into its genome. The virus can be an acutely-transforming or slowly-transforming oncogenic virus. In acutely transforming viruses, the viral particles carry a gene that encodes for an overactive oncogene called viral-oncogene (v-onc), and the infected cell is transformed as soon as v-onc is expressed. In contrast, in slowly-transforming viruses, the virus genome is inserted near a proto-oncogene in the host genome. Exemplary oncoviruses include Human papillomaviruses (HPV), Hepatitis B (HBV), Hepatitis C (HCV), Human T-lymphotropic virus (HTLV), Kaposi's sarcoma-associated herpesvirus (HHV-8), Merkel cell polyomavirus, Epstein-Barr virus (EBV), Human immunodeficiency virus (HIV), and Human cytomegalovirus (CMV).

As used herein, the “virally infected cell” refers to a cell that has been exposed to or infected with a virus or carries viral genetic material, either RNA or DNA. The virus can be an oncogenic virus or a lytic virus or a latent virus and can cause cancer, immunodeficiency, hepatitis, encephalitis, pneumonitis, respiratory illness, or other disease condition. It has previously been shown that retorviruses, specifically HIV, rely in part upon the base excision repair (BER) pathway for integration into host DNA. The ability of 3E10 to inhibit DNA repair provides a mechanism whereby 3E10 and other anti-DNA antibodies can ameliorate virally caused diseases, in particular, by interfering with DNA repair and thereby by blocking the DNA or RNA metabolism that is part of virus life cycles as well as part of viral infection of a cell.

As used herein, the term “individual,” “host,” “subject,” and “patient” are used interchangeably to refer to any individual who is the target of administration or treatment. The subject can be a vertebrate, for example, a mammal. Thus, the subject can be a human or veterinary patient.

As used herein, the term “therapeutically effective” means that the amount of the composition used is of sufficient quantity to ameliorate one or more causes or symptoms of a disease or disorder. Such amelioration only requires a reduction or alteration, not necessarily elimination. A therapeutically effective amount of a composition for treating cancer is preferably an amount sufficient to cause tumor regression or to sensitize a tumor to radiation or chemotherapy.

As used herein, the term “pharmaceutically acceptable” refers to a material that is not biologically or otherwise undesirable, i.e., the material may be administered to a subject without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

As used herein, the term “inhibit” means to decrease an activity, response, condition, disease, or other biological parameter. This can include, but is not limited to, the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

As used herein, the term “fusion protein” refers to a polypeptide formed by the joining of two or more polypeptides through a peptide bond formed between the amino terminus of one polypeptide and the carboxyl terminus of another polypeptide or through linking of one polypeptide to another through reactions between amino acid side chains (for example disulfide bonds between cysteine residues on each polypeptide). The fusion protein can be formed by the chemical coupling of the constituent polypeptides or it can be expressed as a single polypeptide from a nucleic acid sequence encoding the single contiguous fusion protein. Fusion proteins can be prepared using conventional techniques in molecular biology to join the two genes in frame into a single nucleic acid sequence, and then expressing the nucleic acid in an appropriate host cell under conditions in which the fusion protein is produced.

As used herein, the term “variant” refers to a polypeptide or polynucleotide that differs from a reference polypeptide or polynucleotide, but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

Modifications and changes can be made in the structure of the polypeptides of in disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and cofactors. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly where the biological functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamnine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of this disclosure thus contemplate functional or biological equivalents of a polypeptide as set forth above. In particular, embodiments of the polypeptides can include variants having about 50%, 60%, 70%, 80%, 90%, 95%, 96%, 97%, 98%, 99%, or more sequence identity to the polypeptide of interest.

As used herein, the term “percent (%) sequence identity” is defined as the percentage of nucleotides or amino acids in a candidate sequence that are identical with the nucleotides or amino acids in a reference nucleic acid sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For purposes herein, the % sequence identity of a given nucleotides or amino acids sequence C to, with, or against a given nucleic acid sequence D (which can alternatively be phrased as a given sequence C that has or comprises a certain % sequence identity to, with, or against a given sequence D) is calculated as follows:

100 times the fraction W/Z,

where W is the number of nucleotides or amino acids scored as identical matches by the sequence alignment program in that program's alignment of C and D, and where Z is the total number of nucleotides or amino acids in D. It will be appreciated that where the length of sequence C is not equal to the length of sequence D, the % sequence identity of C to D will not equal the % sequence identity of D to C.

### II. Compositions

A. Active Agents

Antigen binding molecules composed of two more antigen binding antibody fragments and/or antigen binding fusion proteins of the antibody 3E10, or a variant thereof are provided. 3E10 preferentially binds DNA single-strand tails, inhibits key steps in DNA single-strand and double-strand break repair (Hansen, et al., Science Translational Medicine, 4:157ra142 (2012)). The antigen binding molecules disclosed herein typically bind to the epitope of 3E10, and preferably inhibit steps in DNA single-strand and double-strand break repair.

Exemplary fragments and fusions include, but are not limited to, single chain antibodies, single chain variable fragments (scFv), disulfide-linked Fvs (sdFv), Fab′, F(ab′)₂, Fv, and single domain antibody fragments (sdAb).

In preferred embodiments, the antigen binding molecule includes two or more scFv. Each scFv can include one, two, or preferably all three complementarity determining regions (CDRs) of the heavy chain variable region (V_{L}) of 3E10, or a variant thereof. The scFv can include one, two, or preferably all three CDRs of the light chain variable region (V_{L}) of 3E10, or a variant thereof. The antigen binding molecule can include the heavy chain variable region and/or light chain variable region of 3E10, or a variant thereof.

A single chain variable fragment can be created by fusing together the variable domains of the heavy and light chains using a short peptide linker, thereby reconstituting an antigen binding site on a single molecule. Single-chain antibody variable fragments (scFvs) in which the C-terminus of one variable domain is tethered to the N-terminus of the other variable domain via a linker have been developed without significantly disrupting antigen binding or specificity of the binding. The linker is chosen to permit the heavy chain and light chain to bind together in their proper conformational orientation. The linker is usually rich in glycine for flexibility, and typically also includes serine or threonine for solubility. The linker can link, for example, the N-terminus of the V_{H} with the C-terminus of the V_{L}, or vice versa. scFv can also be created directly from subcloned heavy and light chains derived from a hybridoma. Preferably, the scFv retains, or improves or increases the specificity of the original immunoglobulin, while removing of the constant regions and introducing the linker.

Exemplary antigen binding molecules that include two or more single chain variable fragments (scFv) including the light chain variable region (V_{L}) of 3E10, or a variant thereof, and the heavy chain variable region (V_{H}) of 3E10, or a variant thereof of the antibody 3E10 include, but are not limited to, divalent-scFv (di-scFv), trivalent-scFv (tri-scFv), multivalent-scFv (multi-scFv), diabodies, triabodies, tetrabodies, etc., of scFvs.

Divalent single chain variable fragments can be engineered by linking two scFvs. This can be done by producing a single peptide chain with two V_{H} and two V_{L} regions, yielding a di-scFvs referred to as a tandem di-scFv. ScFvs can also be designed with linker peptides that are too short for the two variable regions to fold together (about five amino acids), forcing scFvs to dimerize and form a divalent single chain variable fragment referred to as a diabody. Diabodies have been shown to have dissociation constants up to 40-fold lower than corresponding scFvs, indicating that they have a much higher affinity to their target. Even shorter linkers (one or two amino acids) lead to the formation of trimers (triabodies or tribodies). Tetrabodies have also been produced and have been shown to exhibit an even higher affinity to their targets than diabodies.

The disclosed antigen binding molecules includes antigen binding antibody fragments and fusion proteins of 3E10 and variants thereof that typically bind to the same epitope as monoclonal antibody 3E10. In the most preferred embodiments, the antigen binding molecule is a di-, tri-, or multivalent scFv. Although the antigen binding antibody fragment or fusion protein of the antigen binding molecule can include additional antibody domains (e.g., constant domains, hinge domains, etc.,), it preferably does not. 3E10 binds DNA and inhibits DNA repair, which is synthetically lethal to DNA repair-deficient cells. This function is independent of any 3E10 constant regions. By contrast, non-penetrating antibodies such as cetuximab that target extracellular receptors depend in part on Fc-mediated activation of ADCC and complement to exert an effect on tumors. Elimination of the Fc from non-penetrating antibodies could therefore diminish the magnitude of their effect on tumors, but Fc is not required for 3E10 to have an effect on cancer cells. Therefore, 3E10 fragments or fusions that lack an Fc region should be unable to activate ADCC and complement and therefore carry a lower risk of nonspecific side effects.

B. Single Chain Variable Fragments

The single chain variable fragments disclosed herein typically include antigen binding fragments of 3E10, or a variant thereof. The monoclonal antibody 3E10 and active fragments and exemplary variants thereof that are transported in vivo to the nucleus of mammalian cells without cytotoxic effect are discussed in U.S. Pat. Nos. 4,812,397 and 7,189,396, and U.S. Published Application No. 2014/0050723.

The variable domains differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen binding site of antibodies.

The fragments and fusions of antibodies disclosed herein have bioactivity. The fragments and fusions, whether attached to other sequences or not, can include insertions, deletions, substitutions, or other selected modifications of particular regions or specific amino acids residues, provided the activity of the fragment or fusion is not significantly reduced or impaired compared to the nonmodified antibody or antibody fragment.

1. Heavy Chain Variable Region

For example, an amino acid sequence for the heavy chain variable region of 3E10 is:

(SEQ ID NO: 1)
EVQLVESGGGLVKPGGSRKLSCAASGFTFS [symbol] YGMHWVRQAPEKGLEWV
AYISSGSSTIYYADTVKGRFTISRDNAKNTLFLQMTSLRSEDTAMYYC
ARRGLLLDYWGQGTTLTVSS;

Zack, et al., Immunology and Cell Biology, 72:513-520 (1994); GenBank: L16981.1—Mouse Ig rearranged L-chain gene, partial cds; and GenBank: AAA65679.1—immunoglobulin heavy chain, partial [Mus musculus]). The complementarity determining regions (CDRs) are shown with underlining.

An amino acid sequence for a preferred variant of the heavy chain variable region of 3E10 is:

(SEQ ID NO: 2)
EVQLVESGGGLVKPGGSRKLSCAASGFTFS [symbol] YGMHWVRQAPEKGLEWV
AYISSGSSTIYYADTVKGRFTISRDNAKNTLFLQMTSLRSEDTAMYYC
ARRGLLLDYWGQGTTLTVSS.

The complementarity determining regions (CDRs) are shown with underlining.

Amino acid position 31 of the heavy chain variable region of 3E10 has been determined to be influential in the ability of the antibody and fragments thereof to penetrate nuclei and bind to DNA. For example, D31N mutation (bolded and italicized in SEQ ID NOS:1 and 2) in CDR1 penetrates nuclei and binds DNA with much greater efficiency than the original antibody (Zack, et al., Immunology and Cell Biology, 72:513-520 (1994), Weisbart, et al., J. Autoimmun., 11, 539-546 (1998); Weisbart, Int. J. Oncol., 25, 1867-1873 (2004)).

2. Light Chain Variable Region

An amino acid sequence for the light chain variable region of 3E10 is:

(SEQ ID NO: 3)
DIVLTQSPASLAVSLGQRATISCRASKSVSTSSYSYMHWYQQKPGQPP
KLLIKYASYLESGVPARFSGSGSGTDFTLNIHPVEEEDAATYYCQHSR
EFPWTFGGGTKLEIKRADAAP.

The complementarity determining regions (CDRs) are shown with underlining. Other 3E10 light chain sequences are known in the art. See, for example, Zack, et al., J. Immunol., 15; 154(4):1987-94 (1995); GenBank: L16981.1—Mouse Ig rearranged L-chain gene, partial cds; GenBank: AAA65681.1—immunoglobulin light chain, partial [Mus musculus]).

3. Linkers

The term “linker” as used herein includes, without limitation, peptide linkers. The peptide linker can be any size provided it does not interfere with the binding of the epitope by the variable regions. In some embodiments, the linker includes one or more glycine and/or serine amino acid residues. Monovalent single-chain antibody variable fragments (scFvs) in which the C-terminus of one variable domain are typically tethered to the N-terminus of the other variable domain via a 15 to 25 amino acid peptide or linker. The linker is chosen to permit the heavy chain and light chain to bind together in their proper conformational orientation. Linkers in diabodies, triabodies, etc., typically include a shorter linker than that of a monovalent scFv as discussed above. Di-, tri-, and other multivalent scFvs typically include three or more linkers. The linkers can be the same, or different, in length and/or amino acid composition. Therefore, the number of linkers, composition of the linker(s), and length of the linker(s) can be determined based on the desired valency of the scFv as is known in the art. Preferably the linker(s) allows for or drives formation of a di-, tri-, and other multivalent scFv.

For example, a linker can include 4-8 amino acids. In a particular embodiment, a linker includes the amino acid sequence GQSSRSS (SEQ ID NO:4). In another embodiment, a linker includes 15-20 amino acids, preferably 18 amino acids. In a particular embodiment, the linker includes the amino acid sequence GQSSRSSSGGGSSGGGGS (SEQ ID NO:5).

Other flexible linkers include, but are not limited to, the amino acid sequences Gly-Ser, Gly-Ser-Gly-Ser (SEQ ID NO:6), Ala-Ser, Gly-Gly-Gly-Ser (SEQ ID NO:7), (Gly₄-Ser)₂ (SEQ ID NO:8) and (Gly₄-Ser)₄ (SEQ ID NO:9), and (Gly-Gly-Gly-Gly-Ser)₃. (SEQ ID NO:28).

4. Variants

The scFv can be composed of an antibody fragment or fusion protein including an amino acid sequence of a variable heavy chain and/or variable light chain that is at least 45%, at least 50%, at least 55%, at least 60%, at least 65%, at least 70%, at least 75%, at least 80%, at least 85%, at least 90%, at least 95%, or at least 99% identical to the amino acid sequence of the variable heavy chain and/or light chain of 3E10 (e.g., SEQ ID NO:1 or 2; and/or 3, respectively), and which binds to the epitope of 3E10, is selectively lethal to or selectively increases the radiosensitivity and/or chemosensitivity of cells deficient in DNA repair, or a combination thereof. The scFv can be composed of an antibody fragment or fusion protein that includes a CDR that is at least 45%, at least 50%, at least 55%, at least 60%, at least 65%, at least 70%, at least 75%, at least 80%, at least 85%, at least 90%, at least 95%, or at least 99% identical to the amino acid sequence of a CDR of the variable heavy chain and/or light chain of 3E10 (e.g., SEQ ID NO:1 or 2; and/or 3), and which binds to the epitope of 3E10, is selectively lethal to or selectively increases the radiosensitivity and/or chemosensitivity of cells deficient in DNA repair, or a combination thereof. The determination of percent identity of two amino acid sequences can be determined by BLAST protein comparison. In preferred embodiments, scFv includes one, two, three, four, five, or more preferably, all six of the CDRs of the above-described preferred variable domains and which binds to the epitope of 3E10, is selectively lethal to or selectively increases the radiosensitivity and/or chemosensitivity of cells deficient in DNA repair, or a combination thereof.

Predicted complementarity determining regions (CDRs) of the heavy chain variable sequence for 3E10 are known in the art, see, for example, Zack, et al., Immunology and Cell Biology, 72:513-520 (1994) and GenBank Accession number AAA65679.1. Predicted complementarity determining regions (CDRs) of the light chain variable sequence for 3E10 are known in the art, see, for example, GenBank: AAA65681.1—immunoglobulin light chain, partial [Mus musculus].

In some embodiments, antibody fragment or fusion protein is modified to alter its half-life. In some embodiments, it is desirable to increase the half-life of the antibody fragment or fusion protein so that it is present in the circulation or at the site of treatment for longer periods of time. For example, where the antibody fragments or fusion proteins are being used alone to treat cancer, e.g., cancer cells having impaired DNA repair, it may be desirable to maintain titers of the antibody fragment or fusion protein in the circulation or in the location to be treated for extended periods of time. In other embodiments, the half-life of the antibody fragment or fusion protein is decreased to reduce potential side effects. For example, where the antibody fragment or fusion protein is being used in conjunction with radiotherapy or chemotherapy, the antibody fragment or fusion protein is preferably present in the circulation at high doses during the treatment with radiation or antineoplastic drug but is otherwise quickly removed from the circulation. Antibody fragments, such as 3E10 scFv, are expected to have a shorter half-life than full size antibodies. Other methods of altering half-life are known and can be used in the described methods. For example, antibody fragments and fusion proteins can be engineered with Fc variants that extend half-life, e.g., using Xtend™ antibody half-life prolongation technology (Xencor, Monrovia, Calif.).

5. Additional Domains and Moieties

The antibodies, antigen binding fragments, and fusion proteins disclosed herein can optionally include one or more additional domains, targeting moieties, and/or tags, etc.

a. Protein Transduction Domains

In some embodiments, the antigen binding molecule includes one or more domains for enhancing delivery of the polypeptide across the plasma membrane into the interior of cells. For example, antibody fragments and fusion proteins can be modified to include a protein transduction domain (PTD), also known as a cell penetrating peptide (CPPS). PTDs are known in the art, and include, but are not limited to, small regions of proteins that are able to cross a cell membrane in a receptor-independent mechanism (Kabouridis, P., Trends in Biotechnology (11):498-503 (2003)). Although several of PTDs have been documented, the two most commonly employed PTDs are derived from TAT (Frankel and Pabo, Cell, 55(6):1189-93(1988)) protein of HIV and Antennapedia transcription factor from Drosophila, whose PTD is known as Penetratin (Derossi et al., J. Biol. Chem., 269(14):10444-50 (1994)).

The Antennapedia homeodomain is 68 amino acid residues long and contains four alpha helices. Penetratin is an active domain of this protein which consists of a 16 amino acid sequence derived from the third helix of Antennapedia. TAT protein consists of 86 amino acids and is involved in the replication of HIV-1. The TAT PTD consists of an 11 amino acid sequence domain (residues 47 to 57; YGRKKRRQRRR (SEQ ID NO:10) of the parent protein that appears to be critical for uptake. Additionally, the basic domain Tat(49-57) or RKKRRQRRR (SEQ ID NO:11) has been shown to be a PTD. TAT has been favored for fusion to proteins of interest for cellular import. Several modifications to TAT, including substitutions of Glutatmine to Alanine, i.e., Q→A, have demonstrated an increase in cellular uptake anywhere from 90% (Wender et al., Proc. Natl. Acad. Sci. USA., 97(24):13003-8 (2000)) to up to 33 fold in mammalian cells. (Ho et al., Cancer Res., 61(2):474-7 (2001)) The most efficient uptake of modified proteins was revealed by mutagenesis experiments of TAT-PTD, showing that an 11 arginine stretch was several orders of magnitude more efficient as an intercellular delivery vehicle. Thus, some embodiments include PTDs that are cationic or amphipathic. Additionally exemplary PTDs include, but are not limited to, poly-Arg—RRRRRRR (SEQ ID NO:12); PTD-5—RRQRRTSKLMKR (SEQ ID NO:13); Transportan GWTLNSAGYLLGKINLKALAALAKKIL (SEQ ID NO:14); KALA—WEAKLAKALAKALAKHLAKALAKALKCEA (SEQ ID NO:15); and RQIKIWFQNRRMKWKK (SEQ ID NO:16).

In some embodiments, the fusion protein includes an endosomal escape sequence that improves delivery of the protein to the interior of the cell. Endosomal escape sequences are known in the art, see for example, Barka, et al., Histochem. Cytochem., 48(11):1453-60 (2000) and Wadia and Stan, Nat. Med., 10(3):310-5 (2004).

b. Targeting Signal or Domain

In some embodiments, the antibody fragment or fusion protein is optionally modified to include one or targeting signals or domains. The targeting signal or sequence can be specific for a host, tissue, organ, cell, organelle, an organelle such as the nucleus, or cellular compartment. Moreover, the compositions disclosed here can be targeted to other specific intercellular regions, compartments, or cell types.

In some embodiments, the targeting signal binds to a ligand or receptor which is located on the surface of a target cell such as to bring the fusion protein and cell membranes sufficiently close to each other to allow penetration of the fusion protein into the cell. Additional embodiments are directed to specifically delivering the fusion protein to specific tissue or cell types.

In a preferred embodiment, the targeting molecule is selected from the group consisting of an antibody or antigen binding fragment thereof, an antibody domain, an antigen, a cell surface receptor, a cell surface adhesion molecule, a viral envelope protein and a peptide selected by phage display that binds specifically to a defined cell.

Targeting domains to specific cells can be accomplished by modifying the disclosed antibody fragments or fusion proteins to include specific cell and tissue targeting signals. These sequences target specific cells and tissues, but in some embodiments the interaction of the targeting signal with the cell does not occur through a traditional receptor:ligand interaction. The eukaryotic cell includes a number of distinct cell surface molecules. The structure and function of each molecule can be specific to the origin, expression, character and structure of the cell. Determining the unique cell surface complement of molecules of a specific cell type can be determined using techniques well known in the art. One skilled in the art will appreciate that the tropism of the antibody fragment or fusion protein can be altered by changing the targeting signal.

It is known in the art that nearly every cell type in a tissue in a mammalian organism possesses some unique cell surface receptor or antigen. Thus, it is possible to incorporate nearly any ligand for the cell surface receptor or antigen as a targeting signal. For example, peptidyl hormones can be used a targeting moieties to target delivery to those cells which possess receptors for such hormones. Chemokines and cytokines can similarly be employed as targeting signals to target delivery of the complex to their target cells. A variety of technologies have been developed to identify genes that are preferentially expressed in certain cells or cell states and one of skill in the art can employ such technology to identify targeting signals which are preferentially or uniquely expressed on the target tissue of interest.

In some embodiments, the targeting signal is used to selectively target tumor cells. Tumor cells express cell surface markers which may only be expressed in the tumor or present in non-tumor cells but preferentially presented in tumor cells. Exemplary tumor specific cell surface markers include, but are not limited to, alfa-fetoprotein (AFP), C-reactive protein (CRP), cancer antigen-50 (CA-50), cancer antigen-125 (CA-125) associated with ovarian cancer, cancer antigen 15-3 (CA15-3) associated with breast cancer, cancer antigen-19 (CA-19) and cancer antigen-242 associated with gastrointestinal cancers, carcinoembryonic antigen (CEA), carcinoma associated antigen (CAA), epidermal growth factor receptor (EGFR), HER-2, chromogranin A, epithelial mucin antigen (MC5), human epithelium specific antigen (HEA), Lewis(a)antigen, melanoma antigen, melanoma associated antigens 100, 25, and 150, mucin-like carcinoma-associated antigen, multidrug resistance related protein (MRPm6), multidrug resistance related protein (MRP41), Neu oncogene protein (C-erbB-2), neuron specific enolase (NSE), P-glycoprotein (mdrl gene product), multidrug-resistance-related antigen, p170, multidrug-resistance-related antigen, prostate specific antigen (PSA), CD56, and NCAM. In one embodiment, the targeting signal consists of antibodies which are specific to the tumor cell surface markers.

Additional embodiments are directed to specifically delivering the fusion protein to intracellular compartments or organelles. Eukaryotic cells contain membrane bound structures or organelles. Organelles can have single or multiple membranes and exist in both plant and animal cells. Depending on the function of the organelle, the organelle can consist of specific components such as proteins and cofactors. The polypeptides delivered to the organelle can enhance or contribute to the functioning of the organelle. Preferably, the organelle targeting signal enhances delivery of the antibody fragment or fusion protein to the nucleus. Therefore, in a preferred embodiment, the organelle targeting signal is a nuclear localization signal (NLS). NLS are known in the art and include for example, SV 40 T antigen or a fragment thereof, such as PKKKRKV (SEQ ID NO:17). The NLS can be simple cationic sequences of about 4 to about 8 amino acids, or can be bipartite having two interdependent positively charged clusters separated by a mutation resistant linker region of about 10-12 amino acids. Additional representative NLS include but are not limited to GKKRSKV (SEQ ID NO:18); KSRKRKL (SEQ ID NO:19); KRPAATKKAGQAKKKKLDK (SEQ ID NO:20); RKKRKTEEESPLKDKAKKSK (SEQ ID NO:21); KDCVMNKHHRNRCQYCRLQR (SEQ ID NO:22); PAAKRVKLD (SEQ ID NO:23); and KKYENVVIKRSPRKRGRPRK (SEQ ID NO:24).

c. Purification Tags

Polypeptides can be isolated using, for example, chromatographic methods such as affinity chromatography, ion exchange chromatography, hydrophobic interaction chromatography, DEAE ion exchange, gel filtration, and hydroxylapatite chromatography. In some embodiments, polypeptides can be engineered to contain an additional domain containing amino acid sequence that allows the polypeptides to be captured onto an affinity matrix. For example, an Fc-containing polypeptide in a cell culture supernatant or a cytoplasmic extract can be isolated using a protein A column. In addition, a tag such as c-myc, hemagglutinin, polyhistidine, mannose binding protein (MBP), or Flag™ (Kodak) can be used to aid polypeptide purification. Polypeptide enhancing amino acid sequence such as SUMO/SMT3 can also be added to increase expression of the polypeptide of interest. Such tags can be inserted anywhere within the polypeptide, including at either the carboxyl or amino terminus. In some embodiments, the tag is removed following expression of the polypeptide. Other fusions that can be useful include enzymes that aid in the detection of the polypeptide, such as alkaline phosphatase Immunoaffinity chromatography also can be used to purify polypeptides. Polypeptides can additionally be engineered to contain a secretory signal (if there is not a secretory signal already present) that causes the polypeptide to be secreted by the cells in which it is produced. The secreted polypeptide can then be isolated from the cell media.

The therapeutic function of the antigen binding molecule can be enhanced by coupling the antigen binding molecule with a therapeutic agent. Such coupling of can be achieved by making an immunoconjugate or by making a fusion protein, or by linking the antigen binding molecule to, for example, a nucleic acid such as an siRNA or to a small molecule.

6. Humanized Sequences

Many non-human antibodies (e.g., those derived from mice, rats, or rabbits) are naturally antigenic in humans, and thus can give rise to undesirable immune responses when administered to humans. Therefore, humanized 3E10 antibody fragments and fusions are provided. The humanized antigen binding molecules may lessen the chance that the scFv will evoke an undesirable immune response when administered to a human.

Humanized forms of non-human (e.g., murine) antibodies include chimeric immunoglobulins, immunoglobulin chains or fragments thereof which contain minimal sequence derived from non-human immunoglobulin. Humanized antibodies include human immunoglobulins (recipient antibody) in which residues from a complementarity determining region (CDR) of the recipient antibody are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also contain residues that are found neither in the recipient antibody nor in the imported CDR or framework sequences. In general, the humanized antibody will contain substantially all of at least one, and typically two, variable domains, in which all or substantially all of the CDR regions correspond to those of a non-human immunoglobulin and all or substantially all of the FR regions are those of a human immunoglobulin consensus sequence. A humanized antibody can optimally contain at least a portion of an immunoglobulin constant region (Fc), typically that of a human immunoglobulin.

Methods for humanizing non-human antibodies are well known in the art. Generally, a humanized antibody has one or more amino acid residues introduced into it from a source that is non-human. These non-human amino acid residues are often referred to as “import” residues, which are typically taken from an “import” variable domain. Antibody humanization techniques generally involve the use of recombinant DNA technology to manipulate the DNA sequence encoding one or more polypeptide chains of an antibody molecule. Humanization can be essentially performed by substituting rodent CDRs or CDR sequences for the corresponding sequences of a human antibody. Accordingly, a humanized form of a non-human antibody (or a fragment thereof) is a chimeric antibody or fragment, wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important in order to reduce antigenicity. According to the “best-fit” method, the sequence of the variable domain of a rodent antibody is screened against the entire library of known human variable domain sequences. The human sequence which is closest to that of the rodent is then accepted as the human framework (FR) for the humanized antibody. Another method uses a particular framework derived from the consensus sequence of all human antibodies of a particular subgroup of light or heavy chains. The same framework may be used for several different humanized antibodies.

It is further important that antibodies be humanized with retention of high affinity for the antigen and other favorable biological properties. To achieve this goal, humanized antibodies are preferably prepared by a process of analysis of the parental sequences and various conceptual humanized products using three dimensional models of the parental and humanized sequences. Three dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved. In general, the CDR residues are directly and most substantially involved in influencing antigen binding.

7. Exemplary scFV

The Examples below provide specific exemplary mono-scFv (SEQ ID NO:27), di-scFv (SEQ ID NO:26), and tri-scFv (SEQ ID NO:27), and data illustrating that di-scFv and tri-scFv have improved and additional activities compared to their monovalent counterpart. The subsequences corresponding to the different domains of each of the exemplary fusion proteins are also provided below. One of skill in the art will appreciate that the exemplary fusion proteins, or domains thereof, can be utilized to construct fusion proteins discussed in more detail above. For example, in some embodiments, the di-scFv includes a first scFv including a Vk variable region (e.g., amino acids 5-114 of SEQ ID NO:26, or a functional variant or fragment thereof), linked to a VH variable domain (e.g., amino acids 136-251 of SEQ ID NO:26, or a functional variant or fragment thereof), linked to a second scFv including a Vk variable region (e.g., amino acids 271-380 of SEQ ID NO:26, or a functional variant or fragment thereof), linked to a VH variable domain (e.g., amino acids 402-517 of SEQ ID NO:26, or a functional variant or fragment thereof). In some embodiments, a tri-scFv includes a di-scFv linked to a third scFv domain including a Vk variable region (e.g., amino acids 537-646 of SEQ ID NO:27, or a functional variant or fragment thereof), linked to a VH variable domain (e.g., amino acids 668-783 of SEQ ID NO:27, or a functional variant or fragment thereof).

The Vk variable regions can be linked to VH variable domains by, for example, a linker (e.g., (GGGGS)₃ (SEQ ID NO:28), alone or in combination with a (6 aa) of light chain CH1 (amino acids 115-120 of SEQ ID NO:26). Other suitable linkers are discussed above and known in the art. scFv can be linked by a linker (e.g., human IgG CH1 initial 13 amino acids (252-264) of SEQ ID NO:26), alone or in combination with a swivel sequence (e.g., amino acids 265-270 of SEQ ID NO:26). Other suitable linkers are discussed above and known in the art.

Therefore, a di-scFv can include amino acids 5-517 of SEQ ID NO:26. A tri-scFv can include amino acids 5-783 of SEQ ID NO:27. In some embodiments, the fusion proteins include additional domains. For example, in some embodiments, the fusion proteins include sequences that enhance solubility (e.g., amino acids 1-4 of SEQ ID NO:26). Therefore, in some embodiments, a di-scFv can include amino acids 1-517 of SEQ ID NO:26. A tri-scFv can include amino acids 1-783 of SEQ ID NO:27. In some embodiments that fusion proteins include one or more domains that enhance purification, isolation, capture, identification, separation, etc., of the fusion protein. Exemplary domains include, for example, Myc tag (e.g., amino acids 518-533 of SEQ ID NO:26) and/or a His tag (e.g., amino acids 534-539 of SEQ ID NO:26). Therefore, in some embodiments, a di-scFv can include the amino acid sequence of SEQ ID NO:26. A tri-scFv can include the amino acid sequence of SEQ ID NO:27. Other substitutable domains and additional domains are discussed in more detail above.

C. Formulations

The disclosed antigen binding molecules can be formulated in a pharmaceutical composition with, for example, a pharmaceutically acceptable carrier. The antigen binding molecules may be in solution, emulsions, or suspension (for example, incorporated into microparticles, liposomes, or cells). Typically, an appropriate amount of a pharmaceutically-acceptable salt is used in the formulation to render the formulation isotonic. As used herein, the term “pharmaceutically acceptable carrier” encompasses any of the standard pharmaceutical carriers, such as a phosphate buffered saline solution, water and emulsions such as an oil/water or water/oil emulsion, various types of wetting agents, and others disclosed herein and/or known in the art. Examples of pharmaceutically-acceptable carriers include, but are not limited to, saline, Ringer's solution and dextrose solution. The pH of the solution is preferably from about 5 to about 8, and more preferably from about 7 to about 7.5. Pharmaceutical compositions may include carriers, thickeners, diluents, buffers, preservatives, and surface active agents. Further carriers include sustained release preparations such as semi-permeable matrices of solid hydrophobic polymers containing the antibody, which matrices are in the form of shaped particles, e.g., films, liposomes or microparticles. It will be apparent to those persons skilled in the art that certain carriers may be more preferable depending upon, for instance, the route of administration and concentration of composition being administered. Pharmaceutical compositions may also include one or more active ingredients such as antimicrobial agents, anti-inflammatory agents, and anesthetics.

In exemplary preferred embodiments, the antigen binding molecule is formulated in a pharmaceutical composition that is suitable for administration by parenteral route, especially injectable or infusable preparations, those forms allowing the immediate release or delayed and controlled release of the active ingredient.

The compositions can be administered systemically. Preferably, the composition is delivered in manner such that the active agent contacts target tissues, and does not or only minimally contacts tissue that could cause a toxic or adverse event. In some embodiments, the composition is delivered locally to a tumor to the tumor's microenvironment. For example, in a particular embodiment, the composition is delivered by intratumoral injection.

Drugs can be formulated for immediate release, extended release, or modified release. A delayed release dosage form is one that releases a drug (or drugs) at a time other than promptly after administration. An extended release dosage form is one that allows at least a twofold reduction in dosing frequency as compared to that drug presented as a conventional dosage form (e.g. as a solution or prompt drug-releasing, conventional solid dosage form). A modified release dosage form is one for which the drug release characteristics of time course and/or location are chosen to accomplish therapeutic or convenience objectives not offered by conventional dosage forms such as solutions, ointments, or promptly dissolving dosage forms. Delayed release and extended release dosage forms and their combinations are types of modified release dosage forms.

Formulations are prepared using a pharmaceutically acceptable “carrier” composed of materials that are considered safe and effective and may be administered to an individual without causing undesirable biological side effects or unwanted interactions. The “carrier” is all components present in the pharmaceutical formulation other than the active ingredient or ingredients. The term “carrier” includes, but is not limited to, diluents, binders, lubricants, desintegrators, fillers, and coating compositions.

The compound can be administered to a subject with or without the aid of a delivery vehicle. Appropriate delivery vehicles for the compounds are known in the art and can be selected to suit the particular active agent. For example, in some embodiments, the active agent(s) is incorporated into or encapsulated by, or bound to, a nanoparticle, microparticle, micelle, synthetic lipoprotein particle, or carbon nanotube. For example, the compositions can be incorporated into a vehicle such as polymeric microparticles which provide controlled release of the active agent(s). In some embodiments, release of the drug(s) is controlled by diffusion of the active agent(s) out of the microparticles and/or degradation of the polymeric particles by hydrolysis and/or enzymatic degradation.

Suitable polymers include ethylcellulose and other natural or synthetic cellulose derivatives. Polymers which are slowly soluble and form a gel in an aqueous environment, such as hydroxypropyl methylcellulose or polyethylene oxide, may also be suitable as materials for drug containing microparticles or particles. Other polymers include, but are not limited to, polyanhydrides, poly (ester anhydrides), polyhydroxy acids, such as polylactide (PLA), polyglycolide (PGA), poly(lactide-co-glycolide) (PLGA), poly-3-hydroxybut rate (PHB) and copolymers thereof, poly-4-hydroxybutyrate (P4HB) and copolymers thereof, polycaprolactone and copolymers thereof, and combinations thereof. In some embodiments, both agents are incorporated into the same particles and are formulated for release at different times and/or over different time periods. For example, in some embodiments, one of the agents is released entirely from the particles before release of the second agent begins. In other embodiments, release of the first agent begins followed by release of the second agent before the all of the first agent is released. In still other embodiments, both agents are released at the same time over the same period of time or over different periods of time.

Agents and pharmaceutical compositions thereof can be administered in an aqueous solution, by parenteral injection. The formulation may also be in the form of a suspension or emulsion. In general, pharmaceutical compositions are provided including effective amounts of the active agent(s) and optionally include pharmaceutically acceptable diluents, preservatives, solubilizers, emulsifiers, adjuvants and/or carriers. Such compositions include diluents sterile water, buffered saline of various buffer content (e.g., Tris-HCl, acetate, phosphate), pH and ionic strength; and optionally, additives such as detergents and solubilizing agents (e.g., TWEEN® 20, TWEEN® 80 also referred to as polysorbate 20 or 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

To aid dissolution of antibody fragments or fusion proteins into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential nonionic detergents that could be included in the formulation as surfactants are lauromacrogol 400, polyoxyl 40 stearate, polyoxyethylene hydrogenated castor oil 10, 50 and 60, glycerol monostearate, polysorbate 20, 40, 60, 65 and 80, sucrose fatty acid ester, methyl cellulose and carboxymethyl cellulose. These surfactants could be present in the formulation of the protein or derivative either alone or as a mixture in different ratios. Additives which potentially enhance uptake of peptides are for instance the fatty acids oleic acid, linoleic acid and linolenic acid.

### III. Methods of Use

A. Methods of Treatment

The disclosed antigen binding molecules including antigen binding antibody fragments and/or fusion proteins can be used therapeutically in combination with a pharmaceutically acceptable carrier. In some embodiments, the pharmaceutical composition is a unit dosage containing the antigen binding molecule in a pharmaceutically acceptable excipient, wherein the antigen binding molecule is present in an amount effective to inhibit DNA repair in a cancer or infected cell.

The precise dosage will vary according to a variety of factors such as subject-dependent variables (e.g., age, immune system health, clinical symptoms etc.). Exemplary dosages, symptoms, pharmacologic, and physiologic effects are discussed in more detail below. For example, effective dosages and schedules for administering the compositions may be determined empirically, and making such determinations is within the skill in the art. The dosage ranges for the administration of the compositions are those large enough to impair DNA repair in target cells and/or sensitize the target cells to radiotherapy and/or chemotherapy. The dosage should not be so large as to cause adverse side effects, such as unwanted cross-reactions, anaphylactic reactions, and the like. Generally, the dosage will vary with the age, condition, and sex of the patient, route of administration, whether other drugs are included in the regimen, and the type, stage, and location of the cancer or infection to be treated. The dosage can be adjusted by the individual physician in the event of any counter-indications. Dosage can vary, and can be administered in one or more dose administrations daily, for one or several days. Guidance can be found in the literature for appropriate dosages for given classes of pharmaceutical products. A typical daily dosage of the antibody used alone might range from about 1 μg/kg to up to 200 mg/kg of body weight or more per day, depending on the factors mentioned above.

The timing of the administration of the composition will depend on the formulation and/or route of administration used. In some embodiments, administration of the composition is given as a long-term treatment regimen whereby pharmacokinetic steady state conditions will be reached. In general, by way of example only, dosage forms useful in the disclosed methods can include doses in the range of about 1 mg/kg to about 200 mg/kg, 10 mg/kg to 100 mg/kg, 20 mg/kg to 75 mg/kg, or 30 mg/kg to 60 mg/kg of body weight. In other embodiments, the dosage is about 200 mg/m² to about 1000 mg/m², more preferably about 200, 250, 300, 350, 400, 450, 500, 600, 700, 800, 900, or 1000 mg/m². In some embodiments, the unit dosage is in a unit dosage form for intravenous injection. In some embodiments, the unit dosage is in a unit dosage form for intratumoral injection.

The Examples below show that di-, tri-, and multivalent 3E10 scFv may be more potent, more efficacious, and/or less toxic than 3E10 monoclonal antibody or monovalent 3E10 scFv. Therefore, the dosage of di-, tri-, and multivalent 3E10 scFv may be lower than that of a related monovalent 3E10 scFv while achieving the same result. Additionally or alternatively, the dosage of di-, tri-, and multivalent 3E10 scFv may be able to achieve effects that cannot be achieved at an equivalent dosage of 3E10 monoclonal antibody or monovalent 3E10 scFv. Additionally or alternatively, a higher dosage of di-, tri-, and multivalent 3E10 scFv may be tolerated in a subject than the highest tolerated dosage of 3E10 monoclonal antibody or monovalent 3E10 scFv.

The frequency of administration can be, for example, one, two, three, four or more times daily, weekly, every two weeks, every three weeks, or monthly. In some embodiments, the inhibitor is administered to a subject once every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. In some embodiments, the frequency of administration is once weekly, or is once every two weeks, or is once every four weeks, or is twice every week. In some embodiments, a single administration is effective. In some embodiments two or more administrations are needed.

The pharmaceutical composition may be administered in a number of ways depending on whether local or systemic treatment is desired, and on the area to be treated. For example, the compositions may be administered intravenously, intramuscularly, intrathecally, intraperitoneally, or subcutaneously. The compositions may be administered directly into a tumor or tissue, e.g., stereotactically. In some embodiments, the compositions are administered into the brain or liver by injection or by a surgically implanted shunt.

In preferred embodiments, the pharmaceutical composition is administered to the subject by injection or infusion. In a particular embodiment, the injection is a bolus injection. In another preferred embodiment, the pharmaceutical composition is administered to the subject by intravenous infusion. The infusion can be carried out over, seconds, minutes, or hours, for example, at least 1, 2, 3, 4, 5, 10, 30 or more seconds, at least 5, 10, 15, 30, 45, or 60 minutes, or about 1, 1.5, 2, 3, 4, 5 or more hours.

In some embodiments, the effect of the composition on a subject is compared to a control. For example, the effect of the composition on a particular symptom, pharmacologic, or physiologic indicator can be compared to an untreated subject, or the condition of the subject prior to treatment. In some embodiments, the symptom, pharmacologic, or physiologic indicator is measured in a subject prior to treatment, and again one or more times after treatment is initiated. In some embodiments, the control is a reference level, or average determined based on measuring the symptom, pharmacologic, or physiologic indicator in one or more subjects that do not have the disease or condition to be treated (e.g., healthy subjects). In some embodiments, the effect of the treatment is compared to a conventional treatment that is known the art, such as one of those discussed herein.

1. Cancer

The antigen binding molecules disclosed herein can be used to treat, reduce, and/or prevent cancer in a subject. Therefore, the antigen binding molecules can be administered in an effective amount to treat, reduce, and/or prevent cancer in a subject. The effective amount or therapeutically effective amount of the antigen binding molecule to treat cancer or a tumor thereof is typically a dosage sufficient to reduce or prevent a least one symptom of the cancer, or to otherwise provide a desired pharmacologic and/or physiologic effect. The symptom may be physical, such as tumor burden, or biological such as reducing proliferation or increasing death of cancer cells. In some embodiments, the amount is effective to kill tumor cells or reduce or inhibit proliferation or metastasis of the tumor cells. In some embodiments, the amount is effective to reduce tumor burden. In some embodiments, the amount is effective to reduce or prevent at least one comorbidity of the cancer.

In a mature animal, a balance usually is maintained between cell renewal and cell death in most organs and tissues. The various types of mature cells in the body have a given life span; as these cells die, new cells are generated by the proliferation and differentiation of various types of stem cells. Under normal circumstances, the production of new cells is so regulated that the numbers of any particular type of cell remain constant. Occasionally, though, cells arise that are no longer responsive to normal growth-control mechanisms. These cells give rise to clones of cells that can expand to a considerable size, producing a tumor or neoplasm. A tumor that is not capable of indefinite growth and does not invade the healthy surrounding tissue extensively is benign. A tumor that continues to grow and becomes progressively invasive is malignant. The term cancer typically refers to a malignant tumor. In addition to uncontrolled growth, malignant tumors exhibit metastasis. In this process, small clusters of cancerous cells dislodge from a tumor, invade the blood or lymphatic vessels, and are carried to other tissues, where they continue to proliferate. In this way a primary tumor at one site can give rise to a secondary tumor at another site.

The compositions and methods described herein are useful for treating subjects having benign or malignant tumors by delaying or inhibiting the growth of a tumor in a subject, reducing the growth or size of the tumor, inhibiting or reducing metastasis of the tumor, and/or inhibiting or reducing symptoms associated with tumor development or growth.

Malignant tumors which may be treated can be classified according to the embryonic origin of the tissue from which the tumor is derived. Carcinomas are tumors arising from endodermal or ectodermal tissues such as skin or the epithelial lining of internal organs and glands. The disclosed compositions are particularly effective in treating carcinomas. Sarcomas, which arise less frequently, are derived from mesodermal connective tissues such as bone, fat, and cartilage. The leukemias and lymphomas are malignant tumors of hematopoietic cells of the bone marrow. Leukemias proliferate as single cells, whereas lymphomas tend to grow as tumor masses. Malignant tumors may show up at numerous organs or tissues of the body to establish a cancer.

The disclosed antigen binding molecules can be used to treat cells undergoing unregulated growth, invasion, or metastasis.

Tumor cell hypoxia is now recognized as a problem in cancer therapy because it makes cancer cells resistant to treatment with radiation and some chemotherapeutics. Hypoxia is also known to cause impaired DNA repair in cancer cells. Accordingly, in some embodiments, the disclosed active agents are used as targeted agents for hypoxic tumor cells.

Cancer cells that have impaired DNA repair are particularly good targets for the disclosed antigen binding molecules. In some embodiments, the antigen binding molecules are lethal to cells with impaired DNA repair. In preferred embodiments, the cells are defective in the expression of a gene or in the function of a protein involved in DNA repair, DNA synthesis, or homologous recombination. Exemplary genes and associated products include XRCC1, ADPRT (PARP-1), ADPRTL2, (PARP-2), POLYMERASE BETA, CTPS, MLH1, MSH2, FANCD2, PMS2, p53, p21, PTEN, RPA, RPA1, RPA2, RPA3, XPD, ERCC1, XPF, MMS19, RAD51, RAD51B, RAD51C, RAD51D, DMC1, XRCCR, XRCC3, BRCA1, BRCA2, PALB2, RAD52, RAD54, RAD50, MRE11, NB51, WRN, BLM, KU70, KU80, ATM, ATR CHK1, CHK2, FANG family of genes, FANCA, FANCB, FANCC, FANCD1, FANCD2, FANCE, FANCF, FANCG, FANCL, FANCM, RAD1, and RAD9.

In some embodiments, the defective gene is a tumor suppressor gene.

In some embodiments, the gene is associated with maintenance of chromosomal integrity and/or protection from genotoxic stress. In the most preferred embodiments, the cells are deficient in single and/or double strand break repair.

In preferred embodiments, the cells have one or more mutations in BRCA1, BRCA2, and/or PTEN. Gene mutations, such as BRCA1, BRCA2, PTEN mutations, can be identified using standard PCR, hybridization, or sequencing techniques.

In particular embodiments, the cancer cell is defective in DNA damage repair due to hypoxia.

Therefore, in some embodiments, the antigen binding molecules can be used to treat cancers arising from DNA repair deficient familial syndromes, such as breast, ovarian, and pancreatic cancers. In these embodiments, the anti-DNA antibodies can be effective without radiotherapy or chemotherapy. For example, the antigen binding molecules can be used to treat cancers that are linked to mutations in BRCA1, BRCA2, PALB2, or RAD51B, RAD51C, RAD51D, or related genes. The antigen binding molecules can also be used to treat colon cancers, endometrial tumors, or brain tumors linked to mutations in genes associated with DNA mismatch repair, such as MSH2, MLH1, PMS2, and related genes. The antigen binding molecules can also be used to treat cancers with silenced DNA repair genes, such as BRCA1, MLH1, OR RAD51B, RAD51C, or RAD51D. The antigen binding molecules can also be used to treat cancers associated with chromosomal maintenance or genotoxic stress, for example, cancers in which PTEN is mutated or silences. PTEN is frequently inactivated in many cancers including breast, prostate, glioma, melanoma, and lung cancers. In these preferred embodiments, the ability of the antigen binding molecules to inhibit DNA repair combined with the inherent repair deficiencies or other susceptibilities of these cancers can be sufficient to induce cell death.

A representative but non-limiting list of cancers that the antibodies can be used to treat include cancers of the blood and lymphatic system (including leukemias, Hodgkin's lymphomas, non-Hodgkin's lymphomas, solitary plasmacytoma, multiple myeloma), cancers of the genitourinary system (including prostate cancer, bladder cancer, renal cancer, urethral cancer, penile cancer, testicular cancer,), cancers of the nervous system (including mengiomas, gliomas, glioblastomas, ependymomas) cancers of the head and neck (including squamous cell carcinomas of the oral cavity, nasal cavity, nasopharyngeal cavity, oropharyngeal cavity, larynx, and paranasal sinuses), lung cancers (including small cell and non-small cell lung cancer), gynecologic cancers (including cervical cancer, endometrial cancer, vaginal cancer, vulvar cancer ovarian and fallopian tube cancer), gastrointestinal cancers (including gastric, small bowel, colorectal, liver, hepatobiliary, and pancreatic cancers), skin cancers (including melanoma, squamous cell carcinomas, and basal cell carcinomas), breast cancer (including ductal and lobular cancer and triple negative breast cancers), and pediatric cancers (including neuroblastoma, Ewing's sarcoma, Wilms tumor, medulloblastoma).

In some embodiments, the cancer is a neoplasm or tumor that demonstrates some resistance to radiotherapy or chemotherapy. In particular embodiments, the cancer cell is resistant to radiation or chemotherapy due to hypoxia Cancers that are resistant to radiotherapy using standard methods include, but are not limited to, sarcomas, renal cell cancer, melanoma, lymphomas, leukemias, carcinomas, blastomas, and germ cell tumors.

2. Virally Transformed Cells

In some embodiments, the antigen binding molecules including antibody fragments or fusion proteins can be used to treat virally transformed cells, such as cells infected with an oncovirus. The effective amount or therapeutically effective amount to treat virally transfected cells is typically a dosage sufficient to kill the cells and/or sensitive them to another cytotoxic agent, or to otherwise provide a desired pharmacologic and/or physiologic effect. For example, viral transformation can impose phenotypic changes on cell, such as high saturation density, anchorage-independent growth, loss of contact inhibition, loss of orientated growth, immortalization, and disruption of the cell's cytoskeleton. The persistence of at least part of the viral genome within the cell is required for cell transformation. This may be accompanied by the continual expression from a number of viral genes, such as oncogenes. These genes may interfere with a cell's signaling pathway causing the observed phenotypic changes of the cell. In some cases, the viral genome is inserted near a proto-oncogene in the host genome. The end result is a transformed cell showing increased cell division, which is favorable to the virus. In some embodiments, viral transformation, viral infection, and/or metabolism is dependent upon DNA repair mechanisms. In these embodiments, inhibition of DNA repair using the disclosed antigen binding molecules also inhibits viral transformation, viral infection and/or metabolism in the cell.

In some embodiments, viral transformation, viral infection, and/or metabolism is dependent upon metabolism of the virally encoded RNA or DNA as a part of the virus life cycle, producing intermediates subject to binding and/or inhibition by the disclosed antibody fragments or fusion proteins. In these embodiments, treatment with the disclosed antigen binding molecules also inhibits viral transformation, viral infection and/or metabolism in the cell.

Lentiviruses (such as HIV) have been previously found to be dependent on host BER activity for infection and integration (Yoder et al., PLoS One, 2011 March 6(3) e17862). In addition, the ataxia-telangiectesia-mutated (ATM) DNA-damage response appears to be critical to HIV replication (Lau et al., Nat Cell Biol, 2005 7(5): 493-500). In some embodiments, retroviral (including lentiviruses, HIV) infection and integration is dependent on host DNA repair mechanisms. In these embodiments treatment with the disclosed antigen binding molecules also suppresses viral infection/integration and suppresses re-infection in the viral life cycle.

In some embodiments, lentiviral (HIV) replication is dependent on DNA repair. In these embodiments treatment with the disclosed antigen binding molecules also suppresses viral replication and suppresses re-infection in the viral life cycle. Therefore, the disclosed antigen binding molecules can be used to treat cells infected with a virus, such as an oncovirus. In some embodiments, the antigen binding molecule inhibits viral transformation, replication, metabolism, or a combination thereof. Exemplary viruses that can be affected by disclosed antigen binding molecules include Human papillomaviruses (HPV), Hepatitis B (HBV), Hepatitis C (HCV), Human T-lymphotropic virus (HTLV), Kaposi's sarcoma-associated herpesvirus (HHV-8), Merkel cell polyomavirus, Epstein-Barr virus (EBV), Human immunodeficiency virus (HIV), and Human cytomegalovirus (CMV). The antigen binding molecules may also be used to treat a latent virus. In some embodiments, the failure of infected cells to mount a DNA damage response to viruses, such as HSV-1, contribute to the establishment of latency. These virally infected cells therefore have impaired DNA repair and are susceptible to treatment with the disclosed antigen binding molecules. Exemplary latent viruses include CMV, EBV, Herpes simplex virus (type 1 and 2), and Varicella zoster virus.

The disclosed antigen binding molecules may also be used to treat active viral infections due to viruses that give rise to cancer, immunodeficiency, hepatitis, encephalitis, pneumonitis, respiratory illness, or other disease condition, by virtue of the antigen binding molecules' ability to bind to DNA and to interfere with DNA repair or RNA metabolisms that is part of the virus life cycle.

Representative viruses whose life cycle or symptoms of the resulting infection, that may be affected by administration of the antibodies include Human papillomaviruses (HPV), Hepatitis B (HBV), Hepatitis C (HCV), Human T-lymphotropic virus (HTLV), Kaposi's sarcoma-associated herpesvirus (HHV-8), Merkel cell polyomavirus, Epstein-Barr virus (EBV), Human immunodeficiency virus (HIV), and Human cytomegalovirus (CMV).

TABLE 1
Additional viruses that may be affected by administration of the antigen binding
molecules include parvovirus, poxvirus, herpes virus, and other DNA viruses:

| | | Virion | | Nucleic | |
|---|---|---|---|---|---|
| | Examples | naked/ | Capsid | acid | |
| Virus Family | (common names) | enveloped | Symmetry | type | Group |
| 1. Adenoviridae | Adenovirus, | Naked | Icosahedral | ds | I |
| | Infectious canine | | | | |
| | hepatitis virus | | | | |
| 2. Papillomaviridae | Papillomavirus | Naked | Icosahedral | ds | I |
| | | | | circular | |
| 3. Parvoviridae | Parvovirus B19, | Naked | Icosahedral | ss | II |
| | Canine parvovirus | | | | |
| 4. Herpesviridae | Herpes simplex | Enveloped | Icosahedral | ds | I |
| | virus, varicella- | | | | |
| | zoster virus, | | | | |
| | cytomegalovirus, | | | | |
| | Epstein-Barr virus | | | | |
| 5. Poxviridae | Smallpox virus, | Complex | Complex | ds | I |
| | cow pox virus, | coats | | | |
| | sheep pox virus, | | | | |
| | orf virus, monkey | | | | |
| | pox virus, vaccinia | | | | |
| | virus | | | | |
| 6. Hepadnaviridae | Hepatitis B virus | Enveloped | Icosahedral | circular, | VII |
| | | | | partially | |
| | | | | ds | |
| 7. Polyomaviridae | Polyoma virus; JC | Naked | Icosahedral | ds | I |
| | virus (progressive | | | circular | |
| | multifocal | | | | |
| | leukoencephalopathy) | | | | |

| | |
|---|---|
| 8. Anelloviridae | Torque teno virus |

TABLE 2
RNA viruses that may be affected by administration
of the antigen binding molecules include:

| | | Capsid | | Nucleic | |
|---|---|---|---|---|---|
| | Examples | naked/ | Capsid | acid | |
| Virus Family | (common names) | enveloped | Symmetry | type | Group |
| 1. Reoviridae | Reovirus, Rotavirus | Naked | Icosahedral | ds | III |
| 2. Picornaviridae | Enterovirus, Rhinovirus, | Naked | Icosahedral | ss | IV |
| | Hepatovirus, | | | | |
| | Cardiovirus, | | | | |
| | Aphthovirus, Poliovirus, | | | | |
| | Parechovirus, Erbovirus, | | | | |
| | Kobuvirus, Teschovirus, | | | | |
| | Coxsackie | | | | |
| 3. Caliciviridae | Norwalk virus | Naked | Icosahedral | ss | IV |
| 4. Togaviridae | Rubella virus | Enveloped | Icosahedral | ss | IV |
| 5. Arenaviridae | Lymphocytic | Enveloped | Complex | ss(−) | V |
| | choriomeningitis virus | | | | |
| 6. Flaviviridae | Dengue virus, Hepatitis | Enveloped | Icosahedral | ss | IV |
| | C virus, Yellow fever | | | | |
| | virus | | | | |
| 7. Orthomyxoviridae | Influenzavirus A, | Enveloped | Helical | ss(−) | V |
| | Influenzavirus B, | | | | |
| | Influenzavirus C, | | | | |
| | Isavirus, Thogotovirus | | | | |
| 8. Paramyxoviridae | Measles virus, Mumps | Enveloped | Helical | ss(−) | V |
| | virus, Respiratory | | | | |
| | syncytial virus, | | | | |
| | Rinderpest virus, Canine | | | | |
| | distemper virus | | | | |
| 9. Bunyaviridae | California encephalitis | Enveloped | Helical | ss(−) | V |
| | virus, Hantavirus | | | | |
| 10. Rhabdoviridae | Rabies virus | Enveloped | Helical | ss(−) | V |
| 11. Filoviridae | Ebola virus, Marburg | Enveloped | Helical | ss(−) | V |
| | virus | | | | |
| 12. Coronaviridae | Corona virus | Enveloped | Helical | ss | IV |
| 13. Astroviridae | Astrovirus | Naked | Icosahedral | ss | IV |
| 14. Bornaviridae | Borna disease virus | Enveloped | Helical | ss(−) | V |
| 15. Arteriviridae | Arterivirus, Equine | Enveloped | Icosahedral | ss | IV |
| | Arteritis Virus | | | | |
| 16. Hepeviridae | Hepatitis E virus | Naked | Icosahedral | ss | IV |

Retroviruses may also be affected:
Genus Alpharetrovirus; type species: Avian leukosis virus; others include Rous sarcoma virus
Genus Betaretrovirus; type species: Mouse mammary tumour virus
Genus Gammaretrovirus; type species: Murine leukemia virus; others include Feline leukemia virus
Genus Deltaretrovirus; type species: Bovine leukemia virus; others include the cancer-causing Human T-lymphotropic virus
Genus Epsilonretrovirus; type species: Walleye dermal sarcoma virus Genus Lentivirus; type species: Human immunodeficiency virus 1 and human immunodeficiency virus 2; others include Simian, Feline immunodeficiency viruses
Genus Spumavirus; type species: Simian foamy virus
Family Hepadnaviridae—e.g. Hepatitis B virus

Other viral diseases that may be affected by administration of the disclosed antigen binding molecules include Colorado Tick Fever (caused by Coltivirus, RNA virus), West Nile Fever (encephalitis, caused by a flavivirus that primarily occurs in the Middle East and Africa), Yellow Fever, Rabies (caused by a number of different strains of neurotropic viruses of the family Rhabdoviridae), viral hepatitis, gastroenteritis (viral)-acute viral gastroenteritis caused by Norwalk and Norwalk-like viruses, rotaviruses, caliciviruses, and astroviruses, poliomyelitis, influenza (flu), caused by orthomyxoviruses that can undergo frequent antigenic variation, measles (rubeola), paramyxoviridae, mumps, Respiratory syndromes including viral pneumonia and acute respiratory syndromes including croup caused by a variety of viruses collectively referred to as acute respiratory viruses, and respiratory illness caused by the respiratory syncytial virus (RSV, the most dangerous cause of respiratory infection in young children).

In some embodiments, the disclosed compositions are used to treat or prevent a viral infection or the spread or worsening of a viral infection. For example, in some embodiments, the compositions are used to treat or prevent a viral infection or the spread or worsening of a viral infection in a subject that has been exposed to or is at risk of being exposed to a virus, such as those discussed herein.

B. Combination Therapies

In some embodiments, the disclosed antigen binding molecules including antibody fragments and/or fusion proteins can be used in combination with radiotherapy, chemotherapy, or a combination thereof, to treat any cancer, including carcinomas, gliomas, sarcomas, or lymphomas. In these embodiments, the disclosed antigen binding molecules including antibody fragments and/or fusion proteins can sensitize the cells to the DNA-damaging effects of radiotherapy or chemotherapy.

The disclosed antigen binding molecules can increase a cancer's radiosensitivity or chemosensitivity. Effective doses of chemotherapy and/or radiation therapy may be toxic for certain cancers. In some embodiments, the antigen binding molecules decrease the required effective dose of an anti-neoplastic drug or radiation levels needed to treat a cancer, thereby reducing toxicity of the effective dose. For example, the most commonly used dosage of doxorubicin is 40 to 60 mg/m² IV every 21 to 28 days, or 60 to 75 mg/m² IV once every 21 days. If the patient has a bilirubin level between 1.2 and 3 mg/dL, the dose should be reduced by 50%. If the patient has a bilirubin level between 3.1 and 5.0 mg/dL, the dose should be reduced by 75%. Serious irreversible myocardial toxicity leading to congestive heart failure often unresponsive to cardiac support therapy may be encountered as the total dosage of doxorubicin approaches 450 mg/m². When used in combination with the disclosed antigen binding molecules, doxorubicin dosage may be reduced to decrease myocardial toxicity without a loss in efficacy.

In other embodiments, the disclosed antigen binding molecules may be used with normal doses of drug or radiation to increase efficacy. For example, the antigen binding molecules may be used to potentiate a drug or radiation therapy for a cancer that is drug or radiation resistant. Cancers that are resistant to radiotherapy using standard methods include sarcomas, melanomas, carcinomas, and hypoxic tumors.

1. Radiotherapy

The disclosed antigen binding molecules can be used in combination with radiation therapy. Radiation therapy (a.k.a. radiotherapy) is the medical use of ionizing radiation as part of cancer treatment to control malignant cells. Radiotherapy also has several applications in non-malignant conditions, such as the treatment of trigeminal neuralgia, severe thyroid eye disease, pterygium, pigmented villonodular synovitis, prevention of keloid scar growth, and prevention of heterotopic ossification. In some embodiments, the disclosed antigen binding molecules are used to increase radiosensitivity for a non-malignant condition.

Radiation therapy works by damaging the DNA of dividing cells, e.g., cancer cells. This DNA damage is caused by one of two types of energy, photon or charged particle. This damage is either direct or indirect. Indirect ionization happens as a result of the ionization of water, forming free radicals, notably hydroxyl radicals, which then damage the DNA. For example, most of the radiation effect caused by photon therapy is through free radicals. One of the major limitations of photon radiotherapy is that the cells of solid tumors become deficient in oxygen, and tumor cells in a hypoxic environment may be as much as 2 to 3 times more resistant to radiation damage than those in a normal oxygen environment.

Direct damage to cancer cell DNA occurs through high-LET (linear energy transfer) charged particles such as proton, boron, carbon or neon ions. This damage is independent of tumor oxygen supply because these particles act mostly via direct energy transfer usually causing double-stranded DNA breaks. Due to their relatively large mass, protons and other charged particles have little lateral side scatter in the tissue; the beam does not broaden much, stays focused on the tumor shape and delivers small dose side-effects to surrounding tissue. The amount of radiation used in photon radiation therapy is measured in Gray (Gy), and varies depending on the type and stage of cancer being treated. For curative cases, the typical dose for a solid epithelial tumor ranges from 60 to 70 Gy, while lymphomas are treated with lower doses. Post-operative (adjuvant) doses are typically around 45-60 Gy in 1.8-2 Gy fractions (for breast, head, and neck cancers). Many other factors are considered by radiation oncologists when selecting a dose, including whether the patient is receiving chemotherapy, patient co-morbidities, whether radiation therapy is being administered before or after surgery, and the degree of success of surgery.

The response of a cancer to radiation is described by its radiosensitivity. Highly radiosensitive cancer cells are rapidly killed by modest doses of radiation. These include leukemias, most lymphomas and germ cell tumors. The majority of epithelial cancers are only moderately radiosensitive, and require a significantly higher dose of radiation (60-70 Gy) to achieve a radical cure. Some types of cancer are notably radioresistant, that is, much higher doses are required to produce a radical cure than may be safe in clinical practice. Renal cell cancer and melanoma are generally considered to be radioresistant.

The response of a tumor to radiotherapy is also related to its size. For complex reasons, very large tumors respond less well to radiation than smaller tumors or microscopic disease. Various strategies are used to overcome this effect. The most common technique is surgical resection prior to radiotherapy. This is most commonly seen in the treatment of breast cancer with wide local excision or mastectomy followed by adjuvant radiotherapy. Another method is to shrink the tumor with neoadjuvant chemotherapy prior to radical radiotherapy. A third technique is to enhance the radiosensitivity of the cancer by giving certain drugs during a course of radiotherapy. The disclosed antigen binding molecules can serve this third function. In these embodiments, the antigen binding molecule can increase the cell's sensitivity to the radiotherapy, for example, by at least 10%, 15%, 20%, 25%, 30%, 35%, 40%, 45%, 50%. Moreover, the antigen binding molecule can be combined with one or more additional radiosensitizers. Examples of known radiosensitizers include cisplatin, gemcitabine, 5-fluorouracil, pentoxifylline, vinorelbine, PARP inhibitors, histone deacetylase inhibitors, and proteasome inhibitors.

In other embodiments, the dose of radiation can be reduced by 10%, 15%, 20%, 25%, 30%, 35%, 40%, 45%, 50%, or more when administered in combination with the disclosed antigen binding molecules.

2. Chemotherapeutics

Numerous chemotherapeutics, especially antineoplastic drugs, are available for combination with the disclosed antigen binding molecules. The majority of chemotherapeutic drugs can be divided into alkylating agents, antimetabolites, anthracyclines, plant alkaloids, topoisomerase inhibitors, monoclonal antibodies, and other antitumour agents.

In preferred embodiments, the antineoplastic drug damages DNA or interferes with DNA repair since these activities will synergize most effectively with the disclosed antigen binding molecules. In these embodiments, the antigen binding molecule increases the cell's sensitivity to the chemotherapy, for example, by at least 10%, 15%, 20%, 25%, 30%, 35%, 40%, 45%, 50%. Non-limiting examples of antineoplastic drugs that damage DNA or inhibit DNA repair include carboplatin, carmustine, chlorambucil, cisplatin, cyclophosphamide, dacarbazine, daunorubicin, doxorubicin, epirubicin, idarubicin, ifosfamide, lomustine, mechlorethamine, mitoxantrone, oxaliplatin, procarbazine, temozolomide, and valrubicin. In some embodiments, the antineoplastic drug is temozolomide, which is a DNA damaging alkylating agent commonly used against glioblastomas. In some embodiments, the antineoplastic drug is a PARD inhibitor, which inhibits a step in base excision repair of DNA damage. In some embodiments, the antineoplastic drug is a histone deacetylase inhibitor, which suppresses DNA repair at the transcriptional level and disrupt chromatin structure. In some embodiments, the antineoplastic drug is a proteasome inhibitor, which suppresses DNA repair by disruption of ubiquitin metabolism in the cell. Ubiquitin is a signaling molecule that regulates DNA repair. In some embodiments, the antineoplastic drug is a kinase inhibitor, which suppresses DNA repair by altering DNA damage response signaling pathways.

In other embodiments, the antineoplastic drug complements the antigen binding molecule by targeting a different activity in the cancer cell. In these embodiments, the antineoplastic drug does not inhibit DNA repair or damage DNA.

Examples of antineoplastic drugs that can be combined with the disclosed antigen binding molecules include, but are not limited to, alkylating agents (such as temozolomide, cisplatin, carboplatin, oxaliplatin, mechlorethamine, cyclophosphamide, chlorambucil, dacarbazine, lomustine, carmustine, procarbazine, chlorambucil and ifosfamide), antimetabolites (such as fluorouracil, gemcitabine, methotrexate, cytosine arabinoside, fludarabine, and floxuridine), some antimitotics, and vinca alkaloids such as vincristine, vinblastine, vinorelbine, and vindesine), anthracyclines (including doxorubicin, daunorubicin, valrubicin, idarubicin, and epirubicin, as well as actinomycins such as actinomycin D), cytotoxic antibiotics (including mitomycin, plicamycin, and bleomycin), and topoisomerase inhibitors (including camptothecins such as irinotecan and topotecan and derivatives of epipodophyllotoxins such as amsacrine, etoposide, etoposide phosphate, and teniposide).

In other embodiments, the dose of chemotherapy can be reduced by 10%, 15%, 20%, 25%, 30%, 35%, 40%, 45%, 50%, or more when administered in combination with the disclosed antigen binding molecules.

### EXAMPLES

### Example 1: Di-scFv Penetrates Cell Nuclei

### Materials and Methods

Abbreviations

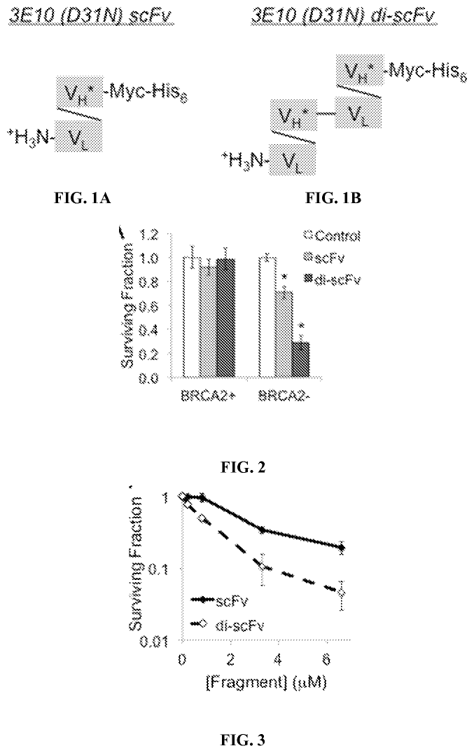

scFv 3E10 (D31N) is a single chain variable fragment including the heavy chain and light chain variable regions of 3E10 and wherein the aspartic acid at position 31 of the heavy chain is mutated to a asparagine (FIG. 1A).

The amino acid sequence for scFv 3E10 (D31N) is:

Annotated Amino Acid Sequence of 3E10 scFv (D31N)

```
(SEQ ID NO: 25)
1 10 20 30 40 50
AGIHDIVLTQSPASLAVSLGQRTISCRASKSVSTSSYSYMHWYQQKPGQP
                  60 70 80 90 100
PKLLIKYASYLESGVPARFSGSGSGTDFTLNIHPVEEEDAATYYCQHSRE
                  110 120 130 140 150
FPWTFGGGTKLEIKRADAAPGGGGSGGGGSGGGGSEVQLVESGGGLVKPG
                  160 170 180 190 200
GSRKLSCAASGFTFSNYGMHWVRQAPEKGLEWVAYISSGSSTIYYADTVK
                  210 220 230 240 250
GRFTISRDNAKNTLFLQMTSLRSEDTAMYYCARRGLLLDYWGQGTTLTVS
                  260 270
SLEQKLISEEDLNSAVDHHHHHH.
```

Annotation of scFv Protein Domains with Reference to SEQ ID NO:25
AGIH sequence increases solubility (amino acids 1-4 of SEQ ID NO:25)
Vk variable region (amino acids 5-114 of SEQ ID NO:25)
Initial (6 aa) of light chain CH1 (amino acids 115-120 of SEQ ID NO:25)
(GGGGS)3 linker (amino acids 121-135 of SEQ ID NO:25)
VH variable region (amino acids 136-251 of SEQ ID NO:25)
Myc tag (amino acids 252-267 of SEQ ID NO:25)
His 6 tag (amino acids 268-273 of SEQ ID NO:25)

Amino Acid Sequence of 3E10 Di-scFv (D31N)

Di-scFv 3E10 (D31N) is a di-single chain variable fragment including 2× the heavy chain and light chain variable regions of 3E10 and wherein the aspartic acid at position 31 of the heavy chain is mutated to a asparagine (FIG. 1B). The amino acid sequence for di-scFv 3E10 (D31N) is:

```
(SEQ ID NO: 26)
1 10 20 30 40 50
AGIHDIVLTQSPASLAVSLGQRTISCRASKSVSTSSYSYMHWYQQKPGQP
                  60 70 80 90 100
PKLLIKYASYLESGVPARFSGSGSGTDFTLNIHPVEEEDAATYYCQHSRE
                  110 120 130 140 150
FPWTFGGGTKLEIKRADAAPGGGGSGGGGSGGGGSEVQLVESGGGLVKPG
                  160 170 180 190 200
GSRKLSCAASGFTFSNYGMHWVRQAPEKGLEWVAYISSGSSTIYYADTVK
                  210 220 230 240 250
GRFTISRDNAKNTLFLQMTSLRSEDTAMYYCARRGLLLDYWGQGTTLTVS
                  260 270 280 290 300
SASTKGPSVFPLAPLESSGSDIVLTQSPASLAVSLGQRTISCRASKSVST
                  310 320 330 340 350
SSYSYMHWYQQKPGQPPKLLIKYASYLESGVPARFSGSGSGTDFTLNIHP
                  360 370 380 390 400
VEEEDAATYYCQHSREFPWTFGGGTKLEIKRADAAPGGGGSGGGGSGGGG
                  410 420 430 440 450
SEVQLVESGGGLVKPGGSRKLSCAASGFTFSNYGMHWVRQAPEKGLEWVA
                  460 470 480 490 500
YISSGSSTIYYADTVKGRFTISRDNAKNTLFLQMTSLRSEDTAMYYCARR
                  510 520 530
GLLLDYWGQGTTLTVSSLEQKLISEEDLNSAVDHHHHHH.
```

Annotation of Di-scFv Protein Domains with Reference to SEQ ID NO:26
AGIH sequence increases solubility (amino acids 1-4 of SEQ ID NO:26)
Vk variable region (amino acids 5-114)
Initial (6 aa) of light chain CH1 (amino acids 115-120 of SEQ ID NO:26)
(GGGGS)3 linker (amino acids 121-135 of SEQ ID NO:26)
VH variable region (amino acids 136-251 of SEQ ID NO:26)
Linker between Fv fragments consisting of human IgG CH1 initial 13 amino acids (amino acids 252-264 of SEQ ID NO:26)
Swivel sequence (amino acids 265-270 of SEQ ID NO:26)
Vk variable region (amino acids 271-380 of SEQ ID NO:26)
Initial (6 aa) of light chain CH1 (amino acids 381-386 of SEQ ID NO:26)
(GGGGS)3 linker (amino acids 387-401 of SEQ ID NO:26)
VH variable region (amino acids 402-517 of SEQ ID NO:26)
Myc tag (amino acids 518-533 of SEQ ID NO:26)
His 6 tag (amino acids 534-539 of SEQ ID NO:26)

Amino Acid Sequence for Tri-scFv

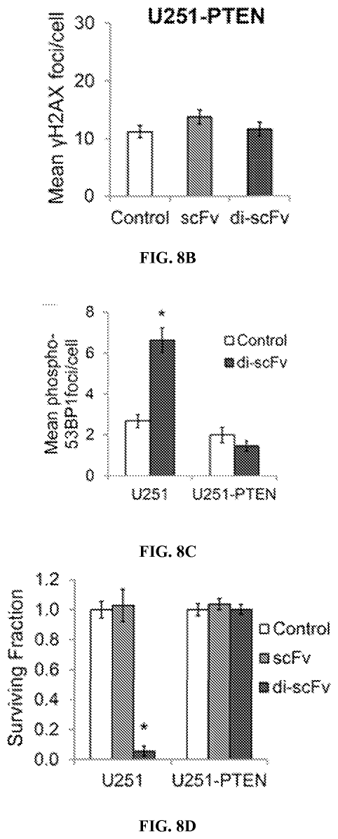

Tri-scFv 3E10 (D31N) is a tri-single chain variable fragment including 3× the heavy chain and light chain variable regions of 310E and wherein the aspartic acid at position 31 of the heavy chain is mutated to a asparagine (FIG. 8). The amino acid sequence for tri-scFv 3E10 (D31N) is:

```
(SEQ ID NO: 27)
1 10 20 30 40 50
AGIHDIVLTQSPASLAVSLGQRTISCRASKSVSTSSYSYMHWYQQKPGQP
                  60 70 80 90 100
PKLLIKYASYLESGVPARFSGSGSGTDFTLNIHPVEEEDAATYYCQHSRE
                  110 120 130 140 150
FPWTFGGGTKLEIKRADAAPGGGGSGGGGSGGGGSEVQLVESGGGLVKPG
                  160 170 180 190 200
GSRKLSCAASGFTFSNYGMHWVRQAPEKGLEWVAYISSGSSTIYYADTVK
                  210 220 230 240 250
GRFTISRDNAKNTLFLQMTSLRSEDTAMYYCARRGLLLDYWGQGTTLTVS
                  260 270 280 290 300
SASTKGPSVFPLAPLESSGSDIVLTQSPASLAVSLGQRTISCRASKSVST
                  310 320 330 340 350
SSYSYMHWYQQKPGQPPKLLIKYASYLESGVPARFSGSGSGTDFTLNIHP
                  360 370 380 390 400
VEEEDAATYYCQHSREFPWTFGGGTKLEIKRADAAPGGGGSGGGGSGGGG
                  410 420 430 440 450
SEVQLVESGGGLVKPGGSRKLSCAASGFTFSNYGMHWVRQAPEKGLEWVA
                  460 470 480 490 500
YISSGSSTIYYADTVKGRFTISRDNAKNTLFLQMTSLRSEDTAMYYCARR
                  510 520 530 540 550
GLLLDYWGQGTTLTVSSASTKGPSVFPLAPLESSGSDIVLTQSPASLAVS
                  560 570 580 590 600
LGQRTISCRASKSVSTSSYSYMHWYQQKPGQPPKLLIKYASYLESGVPAR
                  610 620 630 640 650
FSGSGSGTDFTLNIHPVEEEDAATYYCQHSREFPWTFGGGTKLEIKRADA
                  660 670 680 690 700
APGGGGSGGGGSGGGGSEVQLVESGGGLVKPGGSRKLSCAASGFTFSNYG
                  710 720 730 740 750
MHWVRQAPEKGLEWVAYISSGSSTIYYADTVKGRFTISRDNAKNTLFLQM
                  760 770 780 790 800
TSLRSEDTAMYYCARRGLLLDYWGQGTTLTVSSLEQKLISEEDLNSAVDH
HHHHH.
```

Annotation of Tri-scFv Protein Domains with Reference to SEQ ID NO:27
AGIH sequence increases solubility (amino acids 1-4 of SEQ ID NO:27)
Vk variable region (amino acids 5-114 of SEQ ID NO:27)
Initial (6 aa) of light chain CH1 (amino acids 115-120 of SEQ ID NO:27)
(GGGGS)₃ linker (amino acids 121-135 of SEQ ID NO:27)
VH variable region (amino acids 136-251 of SEQ ID NO:27)
Linker between Fv fragments consisting of human IgG CH1 initial 13 amino acids (amino acids 252-264 of SEQ ID NO:27)
Swivel sequence (amino acids 265-270 of SEQ ID NO:27)
Vk variable region (amino acids 271-380 of SEQ ID NO:27)
Initial (6 aa) of light chain CH1 (amino acids 381-386 of SEQ ID NO:27)
(GGGGS)3 linker (amino acids 387-401 of SEQ ID NO:27)
VH variable region (amino acids 402-517 of SEQ ID NO:27)
Linker between Fv fragments consisting of human IgG CH1 initial 13 amino acids (amino acids 518-530 of SEQ ID NO:27)
Swivel sequence (amino acids 531-536 of SEQ ID NO:27)
Vk variable region (amino acids 537-646 of SEQ ID NO:27)
Initial (6 aa) of light chain CH1 (amino acids 647-652 of SEQ ID NO:27)
(GGGGS)3 linker (amino acids 653-667 of SEQ ID NO:27)
VH variable region (amino acids 668-783 of SEQ ID NO:27)
Myc tag (amino acids 784-799 of SEQ ID NO:27)
His 6 tag (amino acids 800-805 of SEQ ID NO:27)

Recombinant Proteins

ScFv 3E10 (D31N) was generated generally as previously described (Hansen, et al., Science Translational Medicine, 4:157ra142 (2012)). Di-scFv 3E10 (D31N), a 3E10 di-single chain variable fragment with D31N mutations, was generated by recombinant fusion of two 3E10 (D31N) scFv fragments. Tri-scFv, a 3E10 tri-single chain variable fragment with D31N mutations, was generated by recombinant fusion of three 3E10 (D31N) scFv fragments. Intervening linker sequences were placed between scFv 3E10 (D31N) segments in di-scFv 3E10 (D31N) and tri-scFv 3E10 (D31N) as previously described (Weisbart, et al., Int. J. Oncol., 25:1113-1118 (2004)). cDNA encoding di-scFv 3E10 (D31N) or tri-scFv 3E10 (D31N) was ligated into the pPICZαA yeast expression vector followed by transfection into X-33 cells. Di-scFv 3E10 (D31N) and tri-scFv 3E10 (D31N) were expressed in and purified from X-33 supernatant using the techniques as previously described (Hansen, et al., Brain Res., 1088:187-196 (2006); Hansen, et al., J. Biol. Chem., 282:20790-20793 (2007); Zhan, et al., Stroke, 41:538-543 (2010)). Protein concentrations were determined by UV spectrophotometry using a NanoDrop (Thermo Fisher Scientific, Waltham, Mass.).

Cell Lines

A matched pair of isogenic BRCA2-proficient and deficient DLD1 colon cancer cells was obtained from Horizon Discovery Ltd (Cambridge, UK). PTEN-deficient U251, U251 with inducible PTEN (U251-PTEN), and BRCA2-deficient CAPAN-1 cells were a gift from Peter Glazer. H1975, HCT116 and SW48 cells were obtained from Horizon Discovery Ltd. U251 and U251 transfected with a tetracycline-inducible PTEN expression vector system (U251-PTEN) cell lines were grown in DMEM (Life Technologies) supplemented with 10% FBS (FBS; Sigma-Aldrich, Saint Louis, Mo.). PTEN expression was induced in U251-PTEN with the addition of 400 μg/ml G418, and 2 μg/ml blasticidin, 1 μg/ml doxycycline. All other cell lines were grown in RPMI 1640 (Life Technologies) supplemented with 10% fetal bovine serum.

Cell-Penetration Assays

DLD1 cells grown in 96 well plates were treated with control buffer or 5 μM scFv 3E10 (D31N) or di-scFv 3E10 (D31N) or tri-scFv 3E10 (D31N) for one hour at 37° C. Cells were then extensively washed with PBS, fixed with chilled 100% ethanol for 5 minutes, washed again with PBS, and then probed with an anti-Myc antibody (clone 9E10) overnight at 4° C. Cells were then washed and then probed with Alexa488-conjugated goat anti-mouse IgG antibody for 1 hour at 4° C. (Cell Signaling, Danvers, Mass.). Cells were then washed and counterstained with propidium iodide (PI; Sigma-Aldrich) for 30 minutes at room temperature to allow visualization of cell nuclei. Nuclear penetration by the antibodies and PI staining was then imaged using an EVOS fl digital fluorescence microscope (Advanced Microscopy Group, Bothell, Wash.) using green fluorescent protein (GFP) and red fluorescent protein (RFP) filters (40× magnification; Life Technologies). GFP and RFP images were merged using Image J (National Institute of Health, Bethesda, Md.).

Statistics

P values were determined by two-tailed Student's t-test.

### Results

A 3E10 di-single chain variable fragment, referred to herein as di-scFv 3E10 (D31N), was generated (FIGS. 1A and 1B). Purified scFv 3E10 (D31N) and di-scFv 3E10 (D31N) were compared by SDS-PAGE and Western blot. di-scFv 3E10 (D31N) migrated at the expected molecular weight of 60 kDa, and Western blot confirmed the presence of the C-terminal Myc tag, demonstrating that the protein was produced and purified in full-length form.

Di-scFv 3E10 (D31N) penetrates cell nuclei. To confirm that di-scFv 3E10 (D31N) retains the ability to penetrate into cell nuclei, DLD1 colon cancer cells were treated with control buffer or 5 μM scFv 3E10 (D31N) or di-scFv 3E10 (D31N) for one hour. Cells were then washed, fixed, and immunostained for the C-terminal Myc tag in the 3E10 fragments. PI counterstain allowed direct visualization of the location of the nucleus. Fluorescent microscopy revealed that both scFv 3E10 (D31N) and di-scFv 3E10 (D31N) co-localize with the PI stain, demonstrating nuclear localization.

### Example 2: Di-scFv 3E10 (D31N) has a Greater Synthetically Lethal Effect on BRCA2-Deficient Cancer Cells than scFv 3E10 (D31N)

### Materials and Methods

Clonogenic Survival Assays

Surviving fractions of cells treated with control media or media containing scFv 3E10 (D31N) or di-scFv 3E10 (D31N) or tri-scFv 3E10 (D31N) were determined by colony formation assay as previously described (Hansen, et al., Science Translational Medicine, 4:157ra142 (2012)).

Results

The effects of scFv 3E10 (D31N) and di-scFv 3E10 (D31N) were compared on an isogenic pair of BRCA2-proficient and deficient DLD1 colon cancer cells (Hucl, et al., Cancer Res., 68:5023-5030 (2008)). Homology-directed repair (HDR) of DNA double-strand breaks is impaired in the BRCA2-deficient DLD1 cells, which makes them sensitive to inhibitors of base excision repair (BER) or HDR such as 3E10. Cells were treated with control media or media containing 10 μM scFv 3E10 (D31N) or di-scFv 3E10 (D31N), and surviving fractions relative to control were determined by colony formation assay as previously described (Hansen, et al., Science Translational Medicine, 4:157ra142 (2012)). As shown in FIG. 2, scFv 3E10 (D31N) was not significantly toxic to the BRCA2-proficient cells (surviving fraction 0.92±0.05) but reduced the surviving fraction of the BRCA2-deficient cells to 0.71±0.06 (p=0.006). Di-scFv 3E10 (D31N) similarly was not toxic to the BRCA2-proficient cells (surviving fraction 0.99±0.06) but had an even greater impact on the BRCA2-deficient cells than scFv 3E10 (D31N) (surviving fraction 0.29±0.09, p=0.007).

The impact of scFv 3E10 (D31N) and di-scFv 3E10 (D31N) on BRCA2-deficient CAPAN-1 human pancreatic cancer cells was also evaluated. CAPAN-1 cells were treated with scFv 3E10 (D31N) or di-scFv 3E10 (D31N) (0-6.6 μM) and surviving fractions were determined by colony formation assay as previously described. Di-scFv 3E10 (D31N) was again observed to be significantly more toxic to the cells than scFv 3E10 (D31N), and a dose dependent effect was observed (FIG. 3). For example, at a dose as low as 0.8 μM di-scFv 3E10 (D31N) reduced the surviving fraction of the cells to 0.51±0.04, compared to 0.99±0.13 with scFv 3E10 (D31N) (p=0.05). At 6.6 μM, di-scFv 3E10 (D31N) reduced the surviving fraction of the cells to 0.05±0.02 compared to 0.20±0.04 by scFv 3E10 (D31N) (p=0.03). The greater sensitivity of the CAPAN-1 cells to scFv 3E10 (D31N) and di-scFv 3E10 (D31N) compared to the DLD1 cells is consistent with previous reports that the CAPAN-1 cells are particularly sensitive to PARP inhibitors (McCabe, et al., Cancer Biology & Therapy, 4:934-936 (2005)).

### Example 3: Di-scFv 3E10 (D31N) Suppresses the Growth of Subcutaneous CAPAN-1 Xenografts In Vivo

### Materials and Methods

CAPAN-1 Tumor Model

CAPAN-1 tumors were established in athymic (NCr nu/nu) male mice ages 5-6 weeks by subcutaneous injection of 5×10⁶ CAPAN-1 cells in the right flank. 16 mice were injected with tumor cells, and tumors with consistent growth were successfully established in 15 of the mice. One mouse tumor showed early stalling in growth and was excluded from analysis. When tumors reached volume of ˜100 mm³ mice were treated with intraperitoneal injection of di-scFv 3E10 (D31N) (40 mg/kg) (n=8) or an equivalent volume of control PBS (n=7) weekly for three weeks (e.g., days 0, 7, 14). Tumor volumes and mouse body weights were tracked during the experiment, and at completion of the experiment (day 28) mice were sacrificed and tumors were excised and masses recorded.

### Results

The full 3E10 antibody was previously shown to sensitize human glioma xenografts to ionizing radiation in vivo, but the effect of 3E10 or its fragments against DNA repair-deficient tumors in vivo has not previously been tested. CAPAN-1 subcutaneous xenografts were generated in immunodeficient mice, and when tumors reached size of ˜100 mm³ the mice were treated with three weekly intraperitoneal injections of control buffer or di-scFv 3E10 (D31N) (40 mg/kg). Tumor volumes were followed and masses were recorded at the end of the experiment.

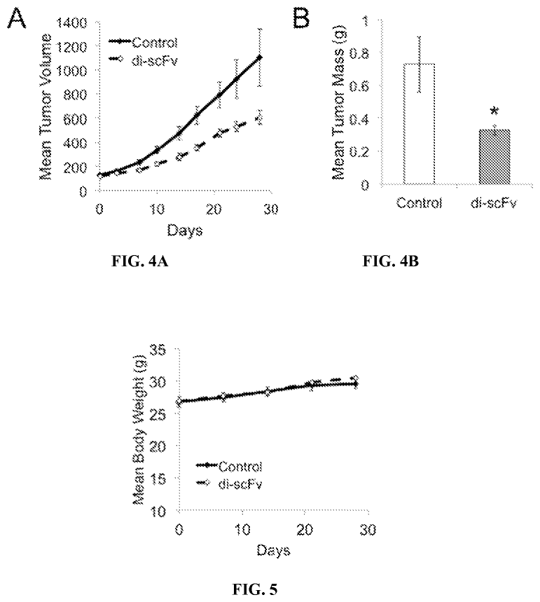

The results show that di-scFv 3E10 (D31N) significantly suppressed the growth of the tumors, as evidenced by decreased tumor volumes and masses (FIGS. 4A and 4B). For example, at the midpoint of the experiment (two weeks after the first treatment) mean tumor volume in mice treated with control buffer was 476±54 mm³ compared to 277±29 mm3 in mice treated with di-scFv 3E10 (D31N) (p=0.005). At the end of the experiment (four weeks after the first treatment) mean tumor volume in mice treated with control buffer was 1102±235 mm³ compared to 606±57 mm³ in mice treated with di-scFv 3E10 (D31N) (p=0.05) (FIG. 4A). Mean tumor mass at the end of the experiment in mice treated with control buffer was 0.73±0.2 g compared to 0.33±0.03 g in mice treated with di-scFv 3E10 (D31N) (p=0.03) (FIG. 4B). Di-scFv 3E10 (D31N) was well tolerated by the mice with no observable toxicities. Mean body weights were indistinguishable between the mice treated with control buffer or di-scFv 3E10 (D31N) (FIG. 5). It is believed that this is the first demonstration of a direct toxic effect of a lupus autoantibody fragment on DNA repair-deficient tumors in vivo.

### Example 4: Tri-scFv 3E10 (D31N) has an Even More Potent Effect on DNA Repair-Deficient Cancer Cells than Di-scFv 3E10 (D31N)

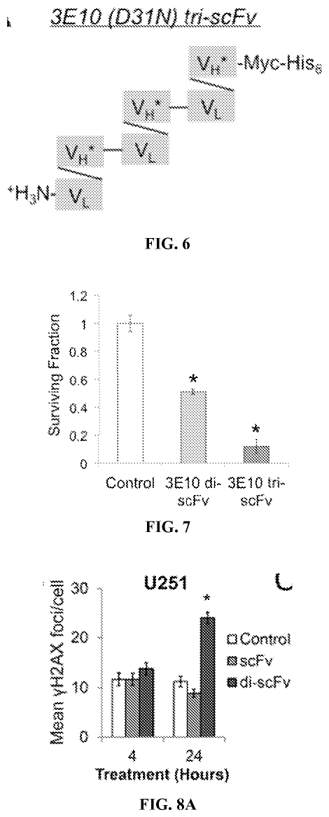

A tri-scFv 3E10 (D31N) with three DNA binding sites was designed, generated, and tested for an effect on DNA repair-deficient cancer cells (FIG. 6). Tri-scFv 3E10 (D31N) was expressed in X-33 and purified from supernatant, similar to scFv 3E10 (D31N) and di-scFv 3E10 (D31N) as discussed above. Total protein stain of the purified tri-scFv 3E10 (D31N) demonstrated isolation of a protein at the expected molecular weight of 90 kDa, and Western blotting for its C-terminal Myc tag confirmed production and purification of full-length tri-scFv 3E10 (D31N).

When tested on cells, tri-scFv 3E10 (D31N) was confirmed by fluorescence microscopy to retain the ability to penetrate into cell nuclei. The impact of tri-scFv 3E10 (D31N) and di-scFv 3E10 (D31N) on the BRCA2-deficient CAPAN-1 cells was also compared. Cells were treated with control media or media containing a low dose (1 μM) of 3E10 (D31N) di-scFv 3E10 (D31N) or tri-scFv 3E10 (D31N). At this low dose di-scFv 3E10 (D31N) still had a significant impact on the cells, with surviving fraction reduced to 0.51±0.02 (p=0.001 relative to control). These data support a conclusion that tri-scFv 3E10 (D31N) is significantly more potent than di-scFv 3E10 (D31N)), because it reduced the surviving fraction to 0.12±0.05 (p<0.0001 relative to control and p=0.002 relative to 3E10 (D31N) di-scFv 3E10 (D31N)) (FIG. 7).

### Example 5: Di-scFv 3E10 (D31N) Causes Accumulation of Detectable Levels of DNA DSBs in PTEN-Deficient Cells

### Material and Methods

γH2AX and phospho-53BP1 foci

U251, U251-PTEN, and CAPAN-1 cells were allowed to adhere to glass coverslips overnight at 37° C. prior and then treated with control buffer or scFv 3E10 (D31N) or di-scFv 3E10 (D31N). Cells were then incubated at 37° C. for 4 or 24 hours and then washed with PBS before fixing with 4% formaldehyde in PBS at 4° C. for 15 minutes. Cells were next washed again and then blocked with 5% goat serum diluted in PBS with 0.3% Triton X-100 for 60 minutes at 4° C. Cells were then incubated overnight at 4° C. with either anti-γH2AX or anti-phospho-53BP1 (Ser1778) (Cell signaling Technology) antibodies. Cells were subsequently washed and probed with Alexa 555-conjugated goat anti-rabbit IgG (H+L) (Life technologies) for 60 minutes at 4° C. Next, cells were washed with PBS and mounted onto microscope slides with ProLong Gold Antifade reagent with DAPI for nuclear counterstaining (Cell Signaling Technology. Slides were cured for 24 hours in the dark before imaging using an Axiovert 200 microscope (Carl Zeiss MicroImaging, Inc.). Mean number of foci per cell was determined from counts in at least 100 cells per condition.

### Results

PTEN-deficiency is associated with numerous malignancies and appears to perturb HDR of DNA DSBs and thereby confer cellular sensitivity to further inhibition of DNA repair by agents such as PARP inhibitors (Puc, et al., Cell cycle, 4:927-9 (2005), Bassi, et al., Science, 341:395-9 (2013), McEllin, et al., Cancer Res., 70:5457-64 (2010)). ScFv 3E10 (D31N) was previously shown to delay resolution of DNA DSBs in irradiated PTEN-deficient U251 cells, as evidenced by persistence of γH2AX foci (a marker of DNA DSBs) in the nuclei of the irradiated cells. However, scFv 3E10 (D31N) has no observable effect on numbers of γH2AX foci in unirradiated U251 cells (Hansen, et al., Science translational medicine, 4:157ra42 (2012)).

Experiments were designed to determine if di-scFv 3E10 (D31N) would have sufficient effect on DNA repair to result in accumulation of detectable levels of DNA DSBs in PTEN-deficient cells even in the absence of IR. U251 cells were treated with control media or 25γM scFv 3E10 (D31N) or di-scFv 3E10 (D31N) for 4 or 24 hours, and γH2AX foci were then visualized by immunofluorescence. scFv 3E10 (D31N) did not yield any increase in γH2AX foci after 4 or 24 hours of treatment, with 11.7±1.3 and 11.2±1.0 mean foci per cell in control cells at 4 and 24 hours, respectively, compared to 11.7±1.2 and 8.8±0.8 mean foci per cell in cells treated with scFv 3E10 (D31N). Di-scFv 3E10 (D31N) similarly did not significantly increase the mean foci per cell after 4 hours of exposure (13.8±1.5 mean foci per cell, p=0.27). However, 24 hours of exposure to di-scFv 3E10 (D31N) resulted in a significant increase in the mean number of foci per cell to 25.1±1.2 (p<0.0001) (FIG. 8A). These data indicate that di-scFv 3E10 (D31N) does not immediately induce DNA damage after penetrating cell nuclei, but instead inhibits DNA repair and causes accumulation of DNA DSBs over a longer period of time, which is consistent with the mechanisms by which 3E10 can inhibit DNA repair (Hansen, et al., Science translational medicine, 4:157ra42 (2012)).

To determine if di-scFv 3E10 (D31N) similarly causes accumulation of DNA DSBs in cells with intact HDR, scFv 3E10 (D31N) and di-scFv 3E10 (D31N) were tested on an isogenic U251 cell line in which PTEN function has been restored (U251-PTEN) (Cleaver, et al., Cell cycle, 10:3223-4 (2011), Levitt, et al., Biochem Biophys Res Commun., 336:1056-61 (2005)). U251-PTEN cells were treated with 25 γM scFv 3E10 (D31N) or di-scFv 3E10 (D31N) for 24 hours and then γH2AX foci were examined as described above. As shown in FIG. 8B, neither scFv 3E10 (D31N) nor di-scFv 3E10 (D31N) caused any significant increase in γH2AX foci in the U251-PTEN cells (11.2±1.1 mean foci per cell in control cells compared to 13.7±1.2 and 11.7±1.2 in cells treated with scFv 3E10 (D31N) and di-scFv 3E10 (D31N), respectively). These data suggest that inhibition of DNA repair by di-scFv 3E10 (D31N) is not sufficient to cause accumulation of DNA DSBs in cells with intact HDR.

p53-binding protein 1 (53BP1) is phosphorylated in response to DNA damage and is recruited to sites of DNA DSBs, and as an additional test to confirm that di-scFv 3E10 (D31N) yields an increase in DNA DSBs in PTEN-deficient cells but not PTEN-proficient cells the effect of di-scFv 3E10 (D31N) on phospho-53BP1 foci in the U251 and U251-PTEN cells was evaluated. The matched pair of cells were treated for 24 hours with control media or 20 μM di-scFv 3E10 (D31N) and then phospho-53BP1 foci were visualized by immunofluorescence. Consistent with the results obtained in the γH2AX assay, di-scFv 3E10 (D31N) significantly increased the mean number of phospho-53BP1 foci in the U251 cells to 6.6±0.6 compared to 2.7±0.3 in control cells, (p<0.0001), but did not increase the number of foci in the U251-PTEN cells (1.5±0.4 foci in control cells and 1.5±0.3 foci in cells treated with di-scFv 3E10 (D31N), p=0.2) (FIG. 8C).

### Example 6: Di-scFv 3E10 (D31N) is Synthetically Lethal to PTEN-Deficient Human Glioma Cells

Unrepaired DNA DSBs are cytotoxic, and based on the differential effect of di-scFv 3E10 (D31N) on the accumulation of DNA DSBs in the U251 and U251-PTEN cells experiments were designed to determine if prolonged exposure to di-scFv 3E10 (D31N) would be toxic to the PTEN-deficient U251 cells but not the U251-PTEN cells. Cells were treated with control media or 3.3 μM scFv 3E10 (D31N) or di-scFv 3E10 (D31N), and surviving fractions were determined by colony formation assay. scFv 3E10 (D31N) was not significantly toxic to either the PTEN-deficient or proficient cells, with surviving fractions of 1.03±0.11 (p=0.81) and 1.04±0.04 (p=0.55), respectively. By contrast, di-scFv 3E10 (D31N) was highly toxic to the PTEN-deficient U251 cells, with surviving fraction reduced to 0.06±0.03 (p<0.0001). The U251-PTEN cells, however, were resistant to di-scFv 3E10 (D31N), with surviving fraction 1.00±0.03, (p=0.98) (FIG. 8D). Taken together, the greater impact of di-scFv 3E10 (D31N) on accumulation of DNA DSBs in and on the survival of PTEN-deficient U251 cells supports the conclusion that di-scFv 3E10 (D31N) has a more potent impact on DNA repair and HDR-deficient cells as compared to scFv 3E10 (D31N) and that di-scFv 3E10 (D31N) has potential as a targeted therapy for HDR-deficient cancer cells.

### Example 7: Di-scFv 3E10 (D31N) Causes Accumulation of DNA DSBs in and is Synthetically Lethal to BRCA2-Deficient Cancer Cells

As described above, PTEN-deficiency is associated with defective HDR of DNA DSBs. HDR is similarly perturbed in cancer cells with defects in BRCA2, and BRCA2-deficient cancer cells are sensitive to inhibitors of base excision repair (BER) or HDR such as 3E10 (Hansen, et al., Science translational medicine, 4:157ra42 (2012), Hucl, et al., Cancer Res., 68:5023-30 (2008), McCabe, et al., Cancer biology & therapy, 4:934-6 (2005)). Experiments were designed to determine if di-scFv 3E10 (D31N) would cause accumulation of DNA DSBs in BRCA2-deficient cells, similar to its effect on PTEN-deficient U251 cells. The impact of di-scFv 3E10 (D31N) on γH2AX foci formation was assessed in BRCA2-deficient CAPAN-1 human pancreatic cancer cells. CAPAN-1 cells were treated with control buffer or 25 μM di-scFv 3E10 (D31N) for 24 hours, and then γH2AX foci were evaluated by immunofluorescence.

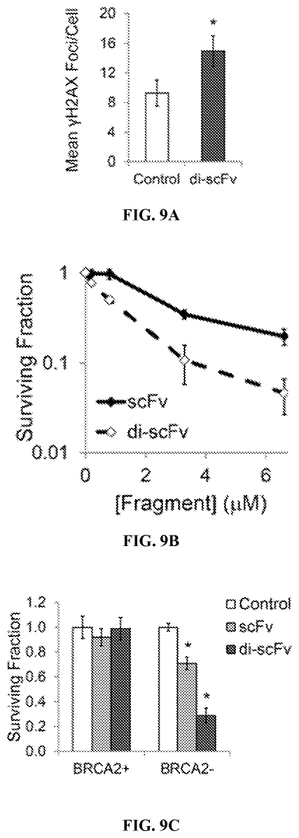

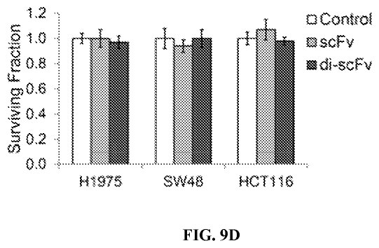

Di-scFv 3E10 (D31N) yielded an increased number of foci (14.9±2.0 foci in cells treated with di-scFv 3E10 (D31N) compared to 9.3±1.8 foci in control cells; p=0.04) (FIG. 9A), indicating accumulation of DNA DSBs similar to the effect observed in the PTEN-deficient U251 cells. Next, the impact of scFv 3E10 (D31N) and di-scFv 3E10 (D31N) on clonogenic survival of CAPAN-1 cells was compared. CAPAN-1 cells were treated with scFv 3E10 (D31N) or di-scFv 3E10 (D31N) (0-6.6 μAA) and surviving fractions were determined by colony formation assay as previously described. Di-scFv 3E10 (D31N) was significantly more toxic to the cells than scFv 3E10 (D31N) (FIG. 9B). For example, at a dose as low as 0.8 μM di-scFv 3E10 (D31N) reduced the surviving fraction of the cells to 0.51±0.04, compared to 0.99±0.13 with scFv 3E10 (D31N) (p=0.05). At 6.6 μM, di-scFv 3E10 (D31N) reduced the surviving fraction of the cells to 0.05±0.02 compared to 0.20±0.04 by scFv 3E10 (D31N) (p=0.03).

To further probe the selectivity of the effect of di-scFv 3E10 (D31N) on HDR-deficient cells di-scFv 3E10 (D31N) was tested on additional cell lines. An isogenic pair of BRCA2-proficient and deficient DLD1 colon cancer cells (Hucl, et al., Cancer Res., 68:5023-30 (2008)) were treated with control media or 10 μM scFv 3E10 (D31N) or di-scFv 3E10 (D31N), and surviving fractions relative to control were determined by colony formation assay. As shown in FIG. 9C, scFv 3E10 (D31N) was not significantly toxic to the BRCA2-proficient cells (surviving fraction 0.92±0.05, p=0.1) but reduced the surviving fraction of the BRCA2-deficient cells to 0.71±0.06 (p=0.006). Di-scFv 3E10 (D31N) similarly was not toxic to the BRCA2-proficient cells (surviving fraction 0.99±0.06, p=0.6) and had a much greater impact on the BRCA2-deficient cells compared to scFv 3E10 (D31N) (surviving fraction 0.29±0.09, p=0.007). ScFv 3E10 (D31N) and di-scFv 3E10 (D31N) were also tested against an additional panel of cells to further confirm that di-scFv 3E10 (D31N) is not simply universally cytotoxic. H1975 (intact DNA repair), SW48 (defective mismatch repair due to MLH1-deficiency), and HCT116 (defective mismatch repair due to MLH1-deficiency) cells were treated with control media or media containing 3.3 μM scFv 3E10 (D31N) or di-scFv 3E10 (D31N), and surviving fractions were determined by colony formation assay. Neither fragment was significantly toxic to any of these cell lines (FIG. 9D). These data demonstrate that di-scFv 3E10 (D31N) is selectively toxic to cells with defects in HDR and is not toxic to cells with intact DNA repair or even to cells with defects in mismatch repair.

3E10 penetrates cells through an equilibrative nucleoside transporter (ENT2) that is expressed in nearly all cells (Hansen, et al., J. Biol. Chem., 282:20790-20793 (2007)), and once inside the nucleus 3E10 appears to incompletely inhibit BER and HDR, which is not toxic to normal cells but is synthetically lethal to cells that have pre-existing defects in DNA repair due to BRCA2-deficiency (Hansen, et al., Science Translational Medicine, 4:157ra142 (2012)). The targeted effect of 3E10 on DNA repair-deficient cancer cells therefore is not due to selective penetration of 3E10 into cancer cells, but rather to the selective sensitivity of DNA repair-deficient cancer cells to further inhibition of DNA repair by 3E10. This is markedly distinct from non-penetrating antibodies that target cancer cells by recognizing and binding to antigens overexpressed on the surface of certain cancer cells (such as EGFR) (Scott, et al., Nature Reviews Cancer, 12:278-287 (2012)).

A further distinction between 3E10 and non-penetrating antibodies is in respective methods of tumor toxicity. 3E10 binds DNA and inhibits DNA repair, which is synthetically lethal to DNA repair-deficient cells. This function is independent of any 3E10 constant regions. By contrast, non-penetrating antibodies such as cetuximab that target extracellular receptors depend in part on Fc-mediated activation of ADCC and complement to exert an effect on tumors (Kellner, et al., Methods, 65:105-113 (2014)). Elimination of the Fc from non-penetrating antibodies would therefore diminish the magnitude of their effect on tumors, however Fc is not required for 3E10 to have an effect on cancer cells. 3E10 fragments that lack an Fc region should be unable to activate ADCC and complement and therefore carry a lower risk of nonspecific side effects.

As described above, scFv 3E10 was previously shown to have only a modest effect on BRCA2-deficient cancer cells at relatively high doses. The data disclosed herein illustrate that di-scFv 3E10 (D31N) has a significantly more potent effect on BRCA2-deficient cancer cells while remaining non-toxic to BRCA2-proficient cells. This is believed to be the first data that di-scFv 3E10 (D31N) suppresses the growth of BRCA2-deficient tumors in vivo and that PTEN-deficient cancer cells are also highly sensitive to di-scFv 3E10 (D31N). PTEN is associated with maintenance of chromosomal integrity, and some PTEN-deficient cancer cells are highly sensitive to genotoxic stress. The observed toxicity of di-scFv 3E10 (D31N) to BRCA2- and PTEN-deficient cells but not cells with intact DNA repair or even with deficient mismatch repair due to MLH1-deficiency is therefore consistent with the conclusion that di-scFv 3E10 (D31N) acts by inhibiting DNA strand break repair. PTEN is frequently inactivated in multiple cancers including breast, prostate, glioma, melanoma, and lung (Bassi, et al., Science, 341:395-399 (2013), Kane, et al., Cancer Res., 57:808-811 (1997), Vlietstra, et al., Cancer Res., 58:2720-2723 (1998), Steck, et al., Nat. Genet., 15:356-362 (1997), Li, et al., Science, 275:1943-1947 (1997)), and the finding that PTEN-deficient cancer cells are sensitive to di-scFv 3E10 (D31N) significantly increases the number of malignancies that may be susceptible to 3E10-based therapy. Therefore, di-scFv 3E10 (D31N) is believed to be a promising targeted therapy for BRCA2- or PTEN-deficient malignancies.

The larger molecular weight of the molecule and its two DNA binding sites results is believed to increase the efficiency of inhibition of DNA repair. The larger size of di-scFv 3E10 (D31N) may contribute to greater steric hindrance of DNA repair factors, and its divalent structure may allow di-scFv 3E10 (D31N) to form DNA crosslinks that are highly toxic to BRCA2- and PTEN-deficient cells. Consistent with this, the data also shows that tri-scFv 3E10 (D31N), with three DNA binding sites and larger molecular weight than di-scFv 3E10 (D31N), has an even greater effect on DNA repair-deficient cells than di-scFv 3E10 (D31N). With a molecular weight of 60 kDa, di-scFv 3E10 (D31N) is also large enough to minimize its renal filtration in vivo, and it is expected that di-scFv 3E10 (D31N) accumulates in tumor tissue in part due to the enhanced permeability and retention (EPR) phenomenon (Jang, et al., Pharm. Res., 20:1337-1350 (2003), Maeda, et al., Journal of Controlled Release: Official Journal of the Controlled Release Society, 65:271-284 (2000)).

1.-2. (canceled)

3. An antigen binding molecule comprising two or more scFv of a humanized variant of monoclonal antibody 3E10, the humanized variant comprising the six complementary determining regions (CDRs) of monoclonal antibody 3E10.

4.-5. (canceled)

6. The antigen binding molecule of claim 3 comprising a heavy chain variable domain comprising the CDRs of SEQ ID NO:1 or SEQ ID NO:2.

7. The antigen binding molecule of claim 6, wherein the heavy chain variable domain comprises SEQ ID NO:1 or SEQ ID NO:2.

8.-9. (canceled)

10. The antigen binding molecule of claim 3 comprising a light chain variable domain comprising the CDRs of SEQ ID NO:3.

11. The antigen binding molecule of claim 10, wherein the light chain variable domain comprises the amino acid sequence of SEQ ID NO:3.

12. The antigen binding molecule of claim 3, wherein the molecule comprises one or more fusion proteins.

13. (canceled)

14. The antigen binding molecule of claim 3, wherein the molecule is a diabody, a tribody, a tetrabody, or a di-scFv.

15.-17. (canceled)

18. The antigen binding molecule of claim 3, wherein the molecule is a tandem di-scFv.

19. The antigen binding molecule of claim 18, wherein the molecule comprises
(i) amino acids 5-517 of SEQ ID NO:26, wherein part or all of amino acids 115-135 of SEQ ID NO:26 and/or amino acids 381-386 of SEQ ID NO:26 are substituted with an alternative linker sequence; and/or wherein amino acids 252-264 (SEQ ID NO:27) are substituted with an alternative linker sequence;
(ii) amino acids 5-517 of SEQ ID NO:26;
(iii) amino acids 1-517 of SEQ ID NO:26;
(iv) amino acids 1-539 of SEQ ID NO:26; or
(v) a functional fragment or variant of any of (i), (ii), (iii), or (iv).

20. (canceled)

21. The antigen binding molecule of claim 3, wherein the molecule is a tandem tri-scFv.

22. The antigen binding molecule of claim 21, wherein the molecule comprises
(i) amino acids 5-517 of SEQ ID NO:26, wherein part or all of amino acids 115-135 of SEQ ID NO:27 and/or amino acids 381-386 of SEQ ID NO:27 and/or amino acids 647-667 of SEQ ID NO:27 are substituted with an alternative linker sequence; and/or wherein amino acids 252-264 (SEQ ID NO:27) and/or amino acids 518-536 (SEQ ID NO:27) are substituted with an alternative linker sequence;
(ii) amino acids 5-783 of SEQ ID NO:27;
(iii) amino acids 1-783 of SEQ ID NO:27;
(iv) amino acids 1-805 of SEQ ID NO:27; or
(v) a functional fragment or variant of any of (i), (ii), (iii), or (iv).

23-25. (canceled)

26. A pharmaceutical composition comprising the antigen binding molecule of claim 3, and pharmaceutically acceptable carrier.

27. A method of inhibiting DNA repair in a neoplastic or virally exposed or infected cell, comprising contacting the cell with a composition comprising an antigen binding molecule comprising two or more scFv of a humanized variant of monoclonal antibody 3E10, the humanized variant comprising the six complementary determining regions (CDRs) of monoclonal antibody 3E10.

28. The method of claim 27, wherein the cell is deficient in DNA damage repair.

29.-31. (canceled)

32. The method of claim 27, wherein the cell has one or more mutations in or abnormal expression of DNA repair genes or impaired function of gene products selected from the group consisting of XRCC1, ADPRT (PARP-1), ADPRTL2, (PARP-2), POLYMERASE BETA, CTPS, MLH1, MSH2, FANCD2, PMS2, p53, p21, PTEN, RPA, RPA1, RPA2, RPA3, XPD, ERCC1, XPF, MMS19, RAD51, RAD51b, RAD51C, RAD51D, DMC1, XRCCR, XRCC3, BRCA1, BRCA2, PALB2, RAD52, RAD54, RAD50, MRE11, NB51, WRN, BLM, KU70, KU80, ATM, ATR CHK1, CHK2, the FANC family of genes, FANCA, FANCB, FANCC, FANCD1, FANCD2, FANCE, FANCF, FANCG, FANCL, FANCM, FANCI, FANCJ, FANCN, FANCP, RAD1, and RAD9.

33. The method of claim 32, wherein the cell is PTEN deficient.

34. The method of claim 27, wherein the cell has a defective tumor suppressor gene, optionally wherein the tumor suppressor gene is BRCA1 or BRCA2.

35.-36. (canceled)

37. The method of claim 27, wherein the neoplastic cell is a cancer cell from a cancer selected from the group consisting of sarcomas, lymphomas, leukemias, carcinomas and adenocarcinomas, blastomas, germ cell tumors, gliomas, neuroendocrine tumors, melanomas, rhabdoid tumors, embryonal tumors, neuroectodermal tumors, carcinoid tumors, craniopharyngiomas, histiocytomas, medulloepitheliomas, mesotheliomas, multiple myelomas, chronic myeloproliferative disease, primitive neuroectodermal tumors, salivary gland tumors, thymomas, thymic carcinoma, thyroid cancer, and Wilms tumor.

38.-57. (canceled)

58. A method of treating cancer in a subject, the method comprising administering to the subject a pharmaceutical composition comprising an antigen binding molecule comprising two or more scFv of a humanized variant of monoclonal antibody 3E10, the humanized variant comprising the six complementary determining regions (CDRs) of monoclonal antibody 3E10.

59. The method of claim 58, further comprising administering the subject a chemotherapeutic or antineoplastic agent.


SEQUENCE LISTING (28 sequences)

SEQ ID NO: 1 - protein, 116 aa, Mus musculus
EVQLVESGGGLVKPGGSRKLSCAASGFTFSDYGMHWVRQAPEKGLEWVAYISSGSSTIYYADTVKGRFTISRDNAKNTLFLQMTSLRSEDTAMYYCARRGLLLDYWGQGTTLTVSS

SEQ ID NO: 2 - protein, 116 aa, Artificial Sequence
  Synthetic Polypeptide - heavy chain variable region
EVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSS

SEQ ID NO: 3 - protein, 117 aa, Mus musculus
DIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIKRADAAP

SEQ ID NO: 4 - protein, 7 aa, Artificial Sequence
  Synthetic Polypeptide - linker
GQSSRSS

SEQ ID NO: 5 - protein, 18 aa, Artificial Sequence
  Synthetic Polypeptide - linker
GQSSRSSSGGGSSGGGGS

SEQ ID NO: 6 - protein, 4 aa, Artificial Sequence
  Synthetic Polypeptide - linker
GSGS

SEQ ID NO: 7 - protein, 4 aa, Artificial Sequence
  Synthetic Polypeptide - linker
GGGS

SEQ ID NO: 8 - protein, 10 aa, Artificial Sequence
  Synthetic Polypeptide - linker
GGGGSGGGGS

SEQ ID NO: 9 - protein, 20 aa, Artificial Sequence
  Synthetic Polypeptide - linker
GGGGSGGGGSGGGGSGGGGS

SEQ ID NO: 10 - protein, 11 aa, Artificial Sequence
  Synthetic Polypeptide - protein transduction domain
YGRKKRRQRRR

SEQ ID NO: 11 - protein, 9 aa, Artificial Sequence
  Synthetic Polypeptide - protein transduction domain
RKKRRQRRR

SEQ ID NO: 12 - protein, 7 aa, Artificial Sequence
  Synthetic Polypeptide - protein transduction domain
RRRRRRR

SEQ ID NO: 13 - protein, 12 aa, Artificial Sequence
  Synthetic Polypeptide - protein transduction domain
RRQRRTSKLMKR

SEQ ID NO: 14 - protein, 27 aa, Artificial Sequence
  Synthetic Polypeptide - protein transduction domain
GWTLNSAGYLLGKINLKALAALAKKIL

SEQ ID NO: 15 - protein, 29 aa, Artificial Sequence
  Synthetic Polypeptide - protein transduction domain
WEAKLAKALAKALAKHLAKALAKALKCEA

SEQ ID NO: 16 - protein, 16 aa, Artificial Sequence
  Synthetic Polypeptide - protein transduction domain
RQIKIWFQNRRMKWKK

SEQ ID NO: 17 - protein, 7 aa, Artificial Sequence
  Synthetic Polypeptide - nuclear localization signal
PKKKRKV

SEQ ID NO: 18 - protein, 7 aa, Artificial Sequence
  Synthetic Polypeptide - nuclear localization signal
GKKRSKV

SEQ ID NO: 19 - protein, 7 aa, Artificial Sequence
  Synthetic Polypeptide - nuclear localization signal
KSRKRKL

SEQ ID NO: 20 - protein, 19 aa, Artificial Sequence
  Synthetic polypeptide - nuclear localization signal
KRPAATKKAGQAKKKKLDK

SEQ ID NO: 21 - protein, 20 aa, Artificial Sequence
  Synthetic Polypeptide - nuclear localization signal
RKKRKTEEESPLKDKAKKSK

SEQ ID NO: 22 - protein, 20 aa, Artificial Sequence
  Synthetic Polypeptide - nuclear localization signal
KDCVMNKHHRNRCQYCRLQR

SEQ ID NO: 23 - protein, 9 aa, Artificial Sequence
  Synthetic Polypeptide - nuclear localization signal
PAAKRVKLD

SEQ ID NO: 24 - protein, 20 aa, Artificial Sequence
  Synthetic Polypeptide - nuclear localization signal
KKYENVVIKRSPRKRGRPRK

SEQ ID NO: 25 - protein, 273 aa, Artificial Sequence
  Synthetic Polypeptide - single chain variable fragment
AGIHDIVLTQSPASLAVSLGQRTISCRASKSVSTSSYSYMHWYQQKPGQPPKLLIKYASYLESGVPARFSGSGSGTDFTLNIHPVEEEDAATYYCQHSREFPWTFGGGTKLEIKRADAAPGGGGSGGGGSGGGGSEVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSSLEQKLISEEDLNSAVDHHHHHH

SEQ ID NO: 26 - protein, 539 aa, Artificial Sequence
  Synthetic Polypeptide - di-single chain variable fragment
AGIHDIVLTQSPASLAVSLGQRTISCRASKSVSTSSYSYMHWYQQKPGQPPKLLIKYASYLESGVPARFSGSGSGTDFTLNIHPVEEEDAATYYCQHSREFPWTFGGGTKLEIKRADAAPGGGGSGGGGSGGGGSEVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSSASTKGPSVFPLAPLESSGSDIVLTQSPASLAVSLGQRTISCRASKSVSTSSYSYMHWYQQKPGQPPKLLIKYASYLESGVPARFSGSGSGTDFTLNIHPVEEEDAATYYCQHSREFPWTFGGGTKLEIKRADAAPGGGGSGGGGSGGGGSEVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSSLEQKLISEEDLNSAVDHHHHHH

SEQ ID NO: 27 - protein, 805 aa, Artificial Sequence
  Synthetic Polypeptide - tri-single chain variable fragment
AGIHDIVLTQSPASLAVSLGQRTISCRASKSVSTSSYSYMHWYQQKPGQPPKLLIKYASYLESGVPARFSGSGSGTDFTLNIHPVEEEDAATYYCQHSREFPWTFGGGTKLEIKRADAAPGGGGSGGGGSGGGGSEVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSSASTKGPSVFPLAPLESSGSDIVLTQSPASLAVSLGQRTISCRASKSVSTSSYSYMHWYQQKPGQPPKLLIKYASYLESGVPARFSGSGSGTDFTLNIHPVEEEDAATYYCQHSREFPWTFGGGTKLEIKRADAAPGGGGSGGGGSGGGGSEVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSSASTKGPSVFPLAPLESSGSDIVLTQSPASLAVSLGQRTISCRASKSVSTSSYSYMHWYQQKPGQPPKLLIKYASYLESGVPARFSGSGSGTDFTLNIHPVEEEDAATYYCQHSREFPWTFGGGTKLEIKRADAAPGGGGSGGGGSGGGGSEVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSSLEQKLISEEDLNSAVDHHHHHH

SEQ ID NO: 28 - protein, 15 aa, Artificial Sequence
  Synthetic Polypeptide - linker
GGGGSGGGGSGGGGS